(19) United States
(12) Patent Application Publication
(10) Pub. No.: US 2024/0293422 A1
(43) Pub. Date: Sep. 5, 2024

(54) SHP2 AND CDK4/6 INHIBITORS COMBINATION THERAPIES FOR THE TREATMENT OF CANCER

(71) Applicant: Erasca, Inc., San Diego, CA (US)

(72) Inventors: Leenus MARTIN, San Diego, CA (US); Leslie Harris BRAIL, San Diego, CA (US); Robert Field SHOEMAKER, San Diego, CA (US)

(21) Appl. No.: 18/572,412

(22) PCT Filed: Jun. 23, 2022
(86) PCT No.: PCT/US2022/034757

§ 371 Date: Dec. 20, 2023

Related U.S. Application Data
(60) Provisional application No. 63/214,744, filed on Jun. 24, 2021.

(51) Int. Cl. A61K 31/5383; A61K 31/453; A61K 31/506; A61K 31/519; A61K 45/06; A61P 35/00

(52) U.S. Cl. CPC A61K 31/5383; A61K 31/453; A61K 31/506; A61K 31/519; A61K 45/06; A61P 35/00

(57) ABSTRACT

The present disclosure provides methods of treating cancer with a combination therapy of a SHP2 inhibitor and an inhibitor of CDK4/6.

### CROSS-REFERENCE

This application claims the benefit of U.S. Provisional Application Ser. No. 63/214,744 filed Jun. 24, 2021, which is hereby incorporated by reference in its entirety.

### BACKGROUND

Src Homology-2 phosphatase (SHP2) is a non-receptor protein phosphatase ubiquitously expressed in various tissues and cell types (see reviews: Tajan M et al., Eur J Med Genet 2016 58(10):509-25; Grossmann K S et al., Adv Cancer Res 2010 106:53-89). SHP2 is composed of two Src homology 2 (N-SH2 and C-SH2) domains in its NH2-terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

The present disclosure provides methods for combination therapy to treat certain cancers using a SHP2 inhibitor in conjunction with a CDK4/6 inhibitor.

### SUMMARY

The present embodiments disclosed herein generally relate to compositions and methods related to combination therapies to treat cancer utilizing a SHP2 inhibitor in conjunction with a CDK4/6 inhibitor, including while providing an unexpected degree synergy.

SHP2 plays important roles in fundamental cellular functions including proliferation, differentiation, cell cycle maintenance and motility. By dephosphorylating its associated signaling molecules, SHP2 regulates multiple intracellular signaling pathways in response to a wide range of growth factors, cytokines, and hormones. Cell signaling processes in which SHP2 participates include the RAS-MAPK (mitogen-activated protein kinase), the PI3K (phosphoinositol 3-kinase)-AKT, and the JAK-STAT pathways.

SHP2 also plays a signal-enhancing role on this pathway, acting downstream of RTKs and upstream of RAS. One common mechanism of resistance involves activation of RTKs that fuel reactivation of the MAPK signaling. RTK activation recruits SHP2 via direct binding and through adaptor proteins. Those interactions result in the conversion of SHP2 from the closed (inactive) conformation to open (active) conformation. SHP2 is an important facilitator of RAS signaling reactivation that bypasses pharmacological inhibition in both primary and secondary resistance. Inhibition of SHP2 achieves the effect of globally attenuating upstream RTK signaling that often drives oncogenic signaling and adaptive tumor escape (see Prahallad, A. et al. Cell Reports 12, 1978-1985 (2015); Chen Y N, Nature 535, 148-152(2016)), which is incorporated herein by reference in its entirety for all of its teachings, including without limitation all methods, compounds, compositions, data, and the like, for use with any of the embodiments and disclosure herein.

In addition to SHP2, the RAS-MAPK signal transduction pathway includes cyclin dependent kinases. Aberrant signaling through cyclin dependent kinases 4/6 (CDK4/6) is a frequent alteration in cancer. Binding of CDK4/6 to cyclin D rather than inhibitor of CDK4 (INK4) proteins phosphorylates retinoblastoma protein (Rb) ultimately resulting in the cell transition from G1 to S phase. Multiple mechanisms increasing singling through CDK4/6 have described in cancer.

KRAS is the most frequently altered RAS isoform and occurs in 86% of pancreatic cancer, 42% of colorectal cancer, and 32% of non-small cell lung cancer (NSCLC). KRAS mutations impaired KRAS' ability to cycle from the GTP-bound active state to the GDP bound inactive state, resulting in an accumulation of KRAS in its active state and oncogenic RAS/MAPK signaling.

In an aspect, provided herein is a method of treating a subject having cancer including administering to the subject a therapeutically effective amount of a compound of Formula I or its pharmaceutically acceptable salt:

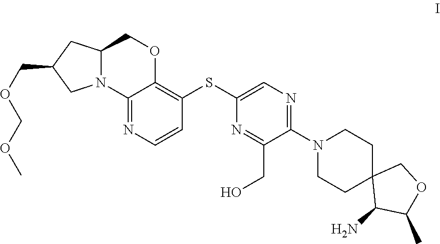 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of cyclin D-cyclin dependent kinase (CDK) 4/6.

In an aspect, provided herein is a method of treating colorectal cancer in a subject including orally administering to the subject a therapeutically effective amount of a compound of Formula I or its pharmaceutically acceptable salt in combination with a CDK4/6 inhibitor.

In embodiments, the cancer is characterized by a mutation in the CDK4/6.

In an aspect, provided herein is a method of treating a subject having cancer comprising:
a) selecting a patient having a cancer characterized by a mutation in the cyclin D-cyclin dependent kinase (CDK) 4/6 pathway; and
b) administering to the subject a therapeutically effective amount of a compound of Formula I or its pharmaceutically acceptable salt:

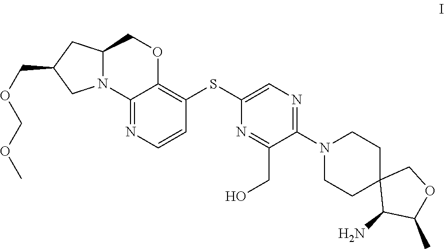 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of CDK4/6.

In embodiments, the mutation in the CDK4/6 pathway includes a mutation in KRAS. In embodiments, the mutation in KRAS is G12D, G12S, G12C, G12V, G13D, Q61H, Q61K, or Q61R.

In embodiments, the cancer is bladder cancer, acute myeloid leukemia, breast cancer, a pan-tumor, a gastrointestinal stromal tumor, colorectal cancer, non-small cell lung cancer, head and neck cancer, endometrial carcinoma, pancreatic cancer, melanoma, head and neck squamous cell carcinoma, liposarcoma, or neuroblastoma. In embodiments, the cancer is colorectal cancer. In embodiments, the cancer is non-small cell lung cancer. In embodiments, the cancer is melanoma. In embodiments, the cancer is breast cancer.

In embodiments, the CDK4/6 inhibitor is palbociclib, ribociclib, abemaciclib, FCN-437c, or alvociclib. In embodiments, the CDK4/6 inhibitor is administered once or twice daily. In embodiments, the dosing of the CDK4/6 inhibitor is in a range from 1 mg to 1000 mg daily. In embodiments, the CDK4/6 inhibitor is administered orally.

In embodiments, the CDK4/6 inhibitor is palbociclib. In embodiments, palbociclib is administered in an amount that is between about 50 mg/day to about 500 mg/day. In embodiments, palbociclib is administered in an amount that is about 50 mg/day, about 75 mg/day, about 100 mg/day, about 125 mg/day, or about 150 mg/day. In embodiments, palbociclib is administered in an amount that is between about 50 mg once a week and about 650 mg once a week. In embodiments, palbociclib is administered in an amount that is about 200 mg once a week, 300 mg once a week, 400 mg once a week, 500 mg once a week, or 600 mg once a week. The amount may be any value or subrange within the recited ranges, including endpoints. In embodiments, palbociclib is administered once a day. In embodiments, palbociclib is administered for three weeks, for example once a day for three weeks. In embodiments, palbociclib is administered for three weeks in a 4 week dosing period. In embodiments, palbociclib is administered for multiple 4 week dosing periods. In embodiments, palbociclib is administered orally.

In embodiments, the CDK4/6 inhibitor is ribociclib. In embodiments, ribociclib is administered in an amount that is between about 50 mg/day to about 1000 mg/day. In embodiments, ribociclib is administered in an amount that is between about 200 mg/day to about 600 mg/day. In embodiments, ribociclib is administered in an amount that is about 50 mg/day, about 100 mg/day, about 200 mg/day, about 400 mg/day, about 500 mg/day, or about 600 mg/day. The amount may be any value or subrange within the recited ranges, including endpoints. In embodiments, ribociclib is administered once a day. In embodiments, ribociclib is administered twice or more per day. In embodiments, ribociclib is administered for three weeks, for example once a day for three weeks. In embodiments, ribociclib is administered for three weeks in a 4 week dosing period. In embodiments, ribociclib is administered for multiple 4 week dosing periods. In embodiments, ribociclib is administered orally.

In embodiments, the CDK4/6 inhibitor is abemaciclib. In embodiments, abemaciclib is administered in an amount that is between about 50 mg/day to about 600 mg/day. In embodiments, abemaciclib is administered in an amount that is between about 150 mg/day to about 400 mg/day. In embodiments, abemaciclib is administered in an amount that is about 75 mg/day, about 150 mg/day, about 200 mg/day, about 300 mg/day, or about 400 mg/day. The amount may be any value or subrange within the recited ranges, including endpoints. In embodiments, ribociclib is administered once a day. In embodiments, abemaciclib is administered twice per day. In embodiments, abemaciclib is administered more than twice per day. In embodiments, abemaciclib is administered daily until disease progression or unacceptable toxicity. In embodiments, abemaciclib is administered orally.

In embodiments, the CDK4/6 inhibitor is FCN-437c. In embodiments, FCN-437c is administered in an amount that is between about 1 mg/day to about 1000 mg/day. In embodiments, FCN-437c is administered in an amount that is between about 50 mg/day to about 800 mg/day. In embodiments, FCN-437c is administered in an amount that is between about 100 mg/day to about 500 mg/day. The amount may be any value or subrange within the recited ranges, including endpoints. In embodiments, FCN-437c is administered once a day. In embodiments, FCN-437c is administered twice per day. In embodiments, FCN-437c is administered more than twice per day. In embodiments, FCN-437c is administered once a week. In embodiments, FCN-437c is administered twice a week. In embodiments, FCN-437c is administered more than twice a week. In embodiments, FCN-437c is administered orally.

In embodiments, the CDK4/6 inhibitor is alvociclib (also called flavopiridol). Ian embodiments, alvociclib is administered in an amount that is between about 1 mg/day to about 1000 mg/day. In embodiments, alvociclib is administered in an amount that is between about 50 mg/day to about 800 mg/day. In embodiments, alvociclib is administered in an amount that is between about 100 mg/day to about 500 mg/day. In embodiments, alvociclib is administered in an amount that is between about 10 mg/m² and about 200 mg/m². In embodiments, alvociclib is administered in an amount that is between about 40 mg/m² and about 150 mg/m². The amount may be any value or subrange within the recited ranges, including endpoints. In embodiments, alvociclib is administered once a day. In embodiments, alvociclib is administered twice per day. In embodiments, alvociclib is administered more than twice per day. In embodiments, alvociclib is administered once a week. In embodiments, alvociclib is administered twice a week. In embodiments, alvociclib is administered more than twice a week. In embodiments, alvociclib is administered orally.

In embodiments, the method includes administering a third MAPK pathway inhibitor. In embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK2 inhibitor, FLT3 inhibitor, or ERK1/2 inhibitor.

In embodiments, the compound of Formula I is administered once or twice daily. In embodiments, the compound of Formula I is administered orally. In embodiments, the dosing of the compound of Formula I is in a range from 20 mg to 400 mg daily.

In embodiments, the compound of Formula I is administered QD or BID for 2 weeks on and 1 week off (21 day schedule).

In embodiments, the compound of Formula I is administered QD or BID for 3 weeks on and 1 week off (28 day schedule).

In embodiments, the compound of Formula I is administered QD or BID three times a week (D1D3D5 TIW) e.g., Day 1, Day 3, and Day 5.

In embodiments, the compound of Formula I is administered twice a day/twice a week e.g., Day 1 and Day 2 (BID-D1D2-BIW).

In embodiments, the compound of Formula I is administered once a day (QD) continuous dosing at a dose of 20 mg/day to 60 mg/day, 40 mg/day, or 60 mg/day.

In embodiments, the compound of Formula I is administered twice a day (BID) continuous dosing at a dose of 20 mg/day to 80 mg/day.

In embodiments, the compound of Formula I is administered twice a day (BID) continuous dosing at a dose of 10 mg/day to 100 mg/day.

In embodiments, the method includes administering a selective estrogen receptor degrader (SERD) or an aromatase inhibitor. In embodiments, the SERD is fulvestrant. In embodiments, the SERD is giredestrant, amcenestrant (SAR439859), AZD9833, rintodestrant, LSZ102, LY3484356, elacestrant, ZN-c5, D-0502, or SHR9549. In embodiments, the aromatase inhibitor is aminoglutethimide, testolactone, anastrozole, letrozole, exemestane, vorozole, formestane, fadrozole, 1,4,6-Androstatrien-3,17-dione (ATD), or 4-Androstene-3,6,17-trione (“6-OXO”).

In an aspect, provided herein is a method of treating a subject having cancer including administering to the subject a therapeutically effective amount of a compound of Formula I or its pharmaceutically acceptable salt in combination with palbociclib.

In an aspect, provided herein is a method of treating a subject having cancer including administering to the subject a therapeutically effective amount of a compound of Formula I or its pharmaceutically acceptable salt in combination with ribociclib.

In an aspect, provided herein is a method of treating a subject having cancer including administering to the subject a therapeutically effective amount of a compound of Formula I or its pharmaceutically acceptable salt in combination with abemaciclib.

In an aspect, provided herein is a method of treating a subject having cancer including administering to the subject a therapeutically effective amount of a compound of Formula I or its pharmaceutically acceptable salt in combination with FCN-437c.

In an aspect, provided herein is a method of treating a subject having cancer including administering to the subject a therapeutically effective amount of a compound of Formula I or its pharmaceutically acceptable salt in combination with alvociclib.

In embodiments, the cancer is colorectal cancer, non-small cell lung cancer, head and neck cancer, endometrial carcinoma, pancreatic cancer, melanoma, head and neck squamous cell carcinoma, liposarcoma, or neuroblastoma. In embodiments, the cancer is colorectal cancer. In embodiments, the cancer is non-small cell lung cancer. In embodiments, the cancer is melanoma. In embodiments, the cancer is breast cancer. In embodiments, the cancer is a pan-tumor.

In embodiments, the subject is a human.

In embodiments, a dosing of the CDK4/6 inhibitor is less than a dosing required for a monotherapy with the CDK4/6 inhibitor. In embodiments, a dosing of the CDK4/6 inhibitor is less than a dosing required for a co-therapy with the CDK4/6 inhibitor and an aromatase inhibitor or a SERD. In embodiments, a dosing of the compound of Formula I is less than a dosing required for a monotherapy with the compound of Formula I.

In an aspect, provided herein is a kit including a compound of Formula I or its pharmaceutically acceptable salt and a CDK4/6 inhibitor. In embodiments, the compound of Formula 1 and the CDK4/6 inhibitor are in separate packages. In embodiments, the CDK4/6 inhibitor is one or more of palbociclib, ribociclib, abemaciclib, FCN-437c, and alvociclib. In embodiments, the kit includes an aromatase inhibitor or a SERD. In embodiments, the kit includes instructions to administer the contents of the kit to a subject for the treatment of cancer.

### BRIEF DESCRIPTION OF THE DRAWINGS

FIG. 1A shows that the combination of the compound of Formula I with palbociclib inhibited of colony formation in KRAS^{G12S} mutated A549 cells (petri dish).

FIG. 1B shows that the combination of the compound of Formula I with palbociclib inhibited of colony formation in KRAS^{G12S} mutated A549 cells (bar graph).

FIG. 2A shows that the combination of the compound of Formula I with palbociclib inhibited colony formation in KRAS^{G12V} mutated NCI-H441 cells (petri dish).

FIG. 2B shows that the combination of the compound of Formula I with palbociclib inhibited colony formation in KRAS^{G12V} mutated NCI-H441 cells (bar graph).

FIG. 3A shows that the combination of the compound of Formula I with palbociclib inhibited colony formation in KRAS^{G12V} mutated SW620 cells (petri dish).

FIG. 3B shows that the combination of the compound of Formula I with palbociclib inhibited colony formation in KRAS^{G12V} mutated SW620 cells (bar graph).

FIG. 4A shows that the combination of the compound of Formula I with palbociclib inhibited colony formation in KRAS^{G12D} mutated LS-180 cells (petri dish).

FIG. 4B shows that the combination of the compound of Formula I with palbociclib inhibited colony formation in KRAS^{G12D} mutated LS-180 cells (bar graph).

FIG. 5A shows that the combination of the compound of Formula I with palbociclib inhibited colony formation in KRAS^{G12D} mutated Gp2D cells (petri dish).

FIG. 5B show that the combination of the compound of Formula I with palbociclib inhibited colony formation in KRAS^{G12D} mutated Gp2D cells (bar graph).

FIG. 6 shows combination efficacy of the compound of Formula I and palbociclib in a KRAS^{G12S} mutant A549 xenograft mouse model. The graph shows the change in tumor volume (mm³) over time (days) under the indicated conditions. Tumor-bearing mice were randomized and treated when mean of tumor volume reached approximately 200 mm³. Mice were dosed orally with indicated dose levels and regimen. Mean of group (n=8) and SEM were calculated and plotted.

FIG. 7 shows mouse body weight change (BWC) in the KRAS^{G12S} mutant NSCLC xenograft model A549. Tumor-bearing mice were dosed orally with indicated dose levels and regimen. Mean of group (n=8) and SEM were calculated and plotted.

FIG. 8 shows that the combination of the compound of Formula I with palbociclib decreased the mean tumor volume in a KRAS^{G13D} NSCLC CDX A-427 xenograft model(CDX=cell-line-derived xenograft).

FIG. 9 shows that the combination of the compound of Formula I with palbociclib decreased the mean tumor volume in a KRAS^{G12D} NSCLC PDX LUN137 xenograft model(PDX=Patient-derived xenograft).

FIG. 10 shows that the combination of the compound of Formula I with palbociclib decreased the mean tumor volume in a KRAS^{G12D} CRC CDX GP2D xenograft model.

FIG. 11 shows that the combination of the compound of Formula I with palbociclib decreased the mean tumor volume in a KRAS^{G12D} CRC CDX LS513 xenograft model.

FIG. 12 shows that the combination of the compound of Formula I with palbociclib decreased the mean tumor volume in a KRAS^{G12V} NSCLC CDX NCI-H441 xenograft model.

### DETAILED DESCRIPTION

The present embodiments provide methods of treating a subject having cancer comprising administering to the subject a therapeutically effective amount of a compound of Formula I or its pharmaceutically acceptable salt:

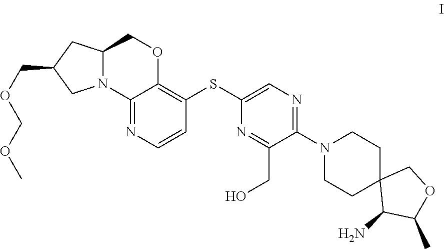 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of a class 1 mutant CDK4/6. The Examples below indicate a significant synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of CDK4/6. Moreover, the combinations of the SHP2 inhibitor of Formula I and inhibitors of CDK4/6 provide methods that allow the use of lower dosages of either agent used alone in a monotherapy, which can aid in reducing potential side effects. Accordingly, such treatments comport with the use of companion diagnostics to aid in proper patient population selection. These and other advantages will be recognized by those skilled in the art.

### Definitions

Unless specifically indicated otherwise, all technical and scientific terms used herein have the same meaning as commonly understood by those of ordinary skill in the art to which the embodiments are directed. In addition, any method or material similar or equivalent to a method or material described herein can be used in the practice of the embodiments herein. For purposes of the embodiments disclosed herein, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, surfactants, coatings, sweeteners, flavors, and colors. One of skill in the art will recognize that other pharmaceutical excipients are useful in the present embodiments.

“Treat,” “treating,” and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. In the context of the combination therapies disclosed herein, administration can be at separate times or simultaneous or substantially simultaneous.

“Co-administering” or “administering in combination with” as used herein refers to administering a composition described herein at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Coadministration is meant to include administration of the compounds on the same day, within the same week, and/or within the same treatment schedule. Compounds may have different administration schedules but still be co-administered if they are administered within the same treatment schedule. For example, palbociclib may be administered once a day for three weeks within a four week treatment schedule, and the compound of Formula I is co-administered with palbociclib if it is administered at any time within the four week treatment schedule.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), each of which is incorporated herein by reference in its entirety for all of its teachings, including without limitation all methods, compounds, compositions, data and the like, for use with any of the embodiments and disclosure herein. In sensitized cells, the therapeutically effective dose can often be lower than the conventional therapeutically effective dose for non-sensitized cells.

“Inhibition,” “inhibits” and “inhibitor” refer to a compound that partially or completely blocks or prohibits or a method of partially or fully blocking or prohibiting, a specific action or function.

“Cancer” refers to all types of cancer, neoplasm or malignant tumors found in mammals (e.g. humans), including, without limitation, leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

### SHP2 Inhibitor

SHP2 plays important roles in fundamental cellular functions including proliferation, differentiation, cell cycle maintenance and motility, and regulates multiple intracellular signaling pathways in response to wide range of growth factors, cytokines, and hormones. Cell signaling processes in which SHP2 participates include MAPK, PI3K and JAK pathways. SHP2 inhibitors have the potential to attenuate upstream RTK signaling that often drives oncogenic signaling and adaptive tumor escape globally, and to become a broad-spectrum anticancer drug.

In some embodiments, the SHP2 inhibitor is Sodium stibogluconate, RMC-4550, NSC87877, SPI-112, TNO155, IACS-13909, GDC01971, or SHP099 HCl. In some embodiments, the SHP2 inhibitor is a compound of Formula I.

### Compound of Formula I

Disclosed herein is (3-((3S,4S)-4-amino-3-methyl-2-oxa-8-azaspiro[4.5]decan-8-yl)-6-(((6aS,8S)-8-((methoxymethoxy)methyl)-6a,7,8,9-tetrahydro-6H-pyrido[3,2-b]pyrrolo[1,2-d] [1,4]oxazin-4-yl)thio)pyrazin-2-yl)methanol

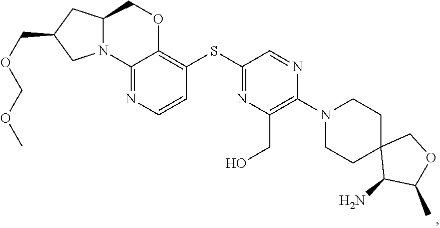 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

### CDK4/6 Inhibitors

CDK4/6 inhibitors act at the G1-to-S cell cycle checkpoint, which is tightly controlled by the D-type cyclins, CDK4 and CDK6. When CDK4 and CDK6 are activated by D-type cyclins, they phosphorylate the retinoblastoma-associated protein (pRb), which releases pRb's suppression of E2F transcription factor family and allow the cell to proceed through cell cycle. In HR+ cancer, cyclin D overexpression is common and loss of pRb is rare, making the G1-to-S checkpoint an ideal therapeutic agent.

In some embodiments, the CDK4/6 inhibitor is palbociclib, ribociclib, abemaciclib, FCN-437c, or alvociclib. In some embodiments, the CDK4/6 inhibitor palbociclib.

### Palbociclib

Palbociclib

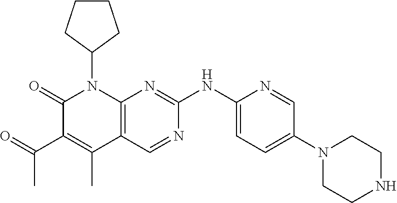 [SMILES: CC(=O)c1c(C)c2cnc(Nc3ccc(N4CCNCC4)cn3)nc2n(C2CCCC2)c1=O]

is a kinase inhibitor used for the treatment of HR+/HER2− advanced or metastatic breast cancer. Palbociclib is sold as Ibrance® by Pfizer.

### COMBINATION METHODS

In embodiments, there are provided methods of treating a subject having cancer comprising administering to the subject a therapeutically effective amount of a compound of Formula I or its pharmaceutically acceptable salt:

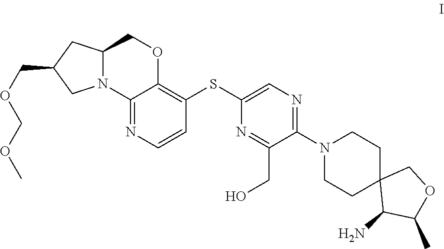 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of CDK4/6. As disclosed herein, a significant synergy was observed beyond that which had been anticipated for such a combination administration.

In an aspect, provided herein is a method of treating a subject having cancer comprising:
a) selecting a patient having a cancer characterized by a mutation in the cyclin D-cyclin dependent kinase (CDK) 4/6 pathway; and
b) administering to the subject a therapeutically effective amount of a compound of Formula I or its pharmaceutically acceptable salt:

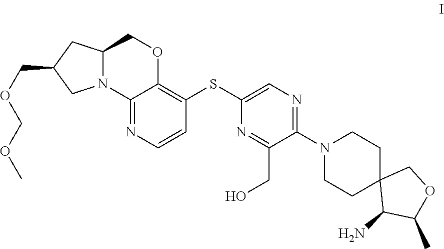 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of CDK4/6.

In embodiments, the methods disclosed herein are suitable for the treatment of any cancer in which there is a mutation in the CDK4/6 pathway. In embodiments, the mutation in the CDK4/6 pathway includes a mutation in KRAS. In embodiments, the mutation in KRAS is G12D, G12S, G12C, G12V, G13D, Q61H, Q61K, or Q61R. In embodiments, the cancer is colorectal cancer, non-small cell lung cancer, head and neck cancer, endometrial carcinoma, pancreatic cancer, melanoma, head and neck squamous cell carcinoma, liposarcoma, or neuroblastoma. In embodiments, the cancer is colorectal cancer. In embodiments, the cancer is non-small cell lung cancer. As will be appreciated by those skilled in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As such, the methods disclosed herein can be used for the treatment of metastases (i.e., metastatic tumor growth) to other organs as well.

Any CDK4/6inhibitor can be used in connection with the present methods. In embodiments, the CDK4/6 inhibitor is palbociclib, ribociclib, abemaciclib, FCN-437c, or alvociclib. In some embodiments, one or more of the inhibitors listed above and elsewhere herein can be specifically excluded from the embodiments set forth herein, including without limitation, any methods, kits, and compositions of matter.

In embodiments, the method comprises administering a third MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In embodiments, any MAPK pathway inhibitor can be employed, including those targeting K-Ras, N-Ras, H-Ras, PDGFRA, PDGFRB, MET, FGFR, ALK, ROS1, TRKA, TRKB, TRKC, EGFR, IGF1R, GRB2, SOS, ARAF, BRAF, RAF1, MEK1, MEK2, c-Myc, CDK4, CDK6, CDK2, ERK1, and ERK2. Exemplary MAPK pathway inhibitors include, without limitation, afatinib, osimertinib, erlotinib, gefitinib, lapatinib, neratinib, dacomitinib, vandetanib, cetuximab, panitumumab, nimotuzumab, necitumumab, trametinib, binimetinib, cobimetinib, selumetinib, ulixertinib, LTT462, and LY3214996.

In some embodiments, the method comprises administering an additional MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, the additional MAPK pathway inhibitor inhibits a protein in the MAPK pathway. In some embodiments, the additional MAPK pathway inhibitor inhibits a protein outside the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, and vandetanib.

In embodiments the additional MAPK pathway inhibitor is adagrasib. In embodiments the additional MAPK pathway inhibitor is afatinib. In embodiments the additional MAPK pathway inhibitors is binimetinib. In embodiments the additional MAPK pathway inhibitor is cetuximab. In embodiments the additional MAPK pathway inhibitor is cobimetinib. In embodiments the additional MAPK pathway inhibitor is dabrafenib. In embodiments the additional MAPK pathway inhibitor is dacomitinib. In embodiments the additional MAPK pathway inhibitor is encorafenib. In embodiments the additional MAPK pathway inhibitor is erlotinib. In embodiments the additional MAPK pathway inhibitor is gefitinib. In embodiments the additional MAPK pathway inhibitor is gilteritinib. In embodiments the additional MAPK pathway inhibitor is lapatinib. In embodiments the additional MAPK pathway inhibitor is LTT462. In embodiments the additional MAPK pathway inhibitor is LY3214996. In embodiments the additional MAPK pathway inhibitor is necitumumab. In embodiments the additional MAPK pathway inhibitor is neratinib. In embodiments the additional MAPK pathway inhibitor is nimotuzumab. In embodiments the additional MAPK pathway inhibitor is osimertinib. In embodiments the additional MAPK pathway inhibitor is panitumumab. In embodiments the additional MAPK pathway inhibitor is selumetinib. In embodiments the additional MAPK pathway inhibitor is sotorasib. In embodiments the additional MAPK pathway inhibitor is trametinib. In embodiments the additional MAPK pathway inhibitor is ulixertinib. In embodiments the additional MAPK pathway inhibitor is vandetanib. In some embodiments, one or more of the MAPK pathway inhibitors listed above and elsewhere herein can be specifically excluded from the embodiments set forth herein, including without limitation, any methods, kits, and compositions of matter.

The methods disclosed herein can be combined with other chemotherapeutic agents. Examples of such agents can be found in Cancer Principles and Practice of Oncology by V. T. Devita and S. Hellman (editors), 6th edition (Feb. 15, 2001), Lippincott Williams & Wilkins Publishers; which is incorporated herein by reference in its entirety for all of its teachings, including without limitation all methods, compounds, compositions, data, and the like, for use with any of the embodiments and disclosure herein. A person of ordinary skill in the art would be able to discern which combinations of agents would be useful based on the particular characteristics of the drugs and the disease involved.

In embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data, and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Each of the above-described references is incorporated herein by reference in its entirety for all of its teachings, including without limitation all methods, compounds, compositions, data, and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof, autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic, and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In embodiments, the dosing of the compound of Formula I can be in any suitable amount to treat the cancer. For example, the dosing could be a daily dosage of between 1 mg up to 500 mg. As an additional example, the daily dose could be in a range from about 20 mg to 400 mg (or any sub-range or sub-value there between, including endpoints). In some embodiments, the range of dosing of the compound of Formula I can be from 10 mg to 300 mg. In some embodiments, the range of dosing of the compound of Formula I can be from 10 mg to 100 mg. In some embodiments, the range of dosing of the compound of Formula I can be from 5 mg to 50 mg. The daily dosage can be achieved by administering a single administered dosage (e.g., QD) or via multiple administrations during a day (e.g., BID, TID, QID, etc.) to provide the total daily dosage.

In some embodiments, the compound of Formula I is administered QD or BID for 2 weeks on and 1 week off (21 day schedule). In some embodiments, the compound of Formula I is administered QD or BID for 3 weeks on and 1 week off (28 day schedule). In some embodiments, the compound of Formula I is administered QD or BID three times a week (D1D3D5 TIW) e.g., Day 1, Day 3, and Day 5. In some embodiments, the compound of Formula I is administered twice a day/twice a week e.g., Day 1 and Day 2 (BID-D1D2-BIW).

In some embodiments, the compound of Formula I is administered once a day (QD) continuous dosing at a dose of 20 mg/day to 60 mg/day, 40 mg/day, or 60 mg/day. In some embodiments, the compound of Formula I is administered twice a day (BID) continuous dosing at a dose of 20 mg/day to 80 mg/day. In some embodiments, the compound of Formula I is administered twice a day (BID) continuous dosing at a dose of 10 mg/day to 100 mg/day.

In embodiments, the dosing of the CDK4/6 inhibitor is any suitable amount. For example, it can be an amount in a range from 1 mg to 1000 mg daily (or any sub-range or sub-value there between, including endpoints). Dosing of the CDK4/6 inhibitor may be the same or less than the approved dosing for any given CDK4/6 inhibitor and may depend on a given indication. In embodiments, palbociclib may be administered from 50 mg to 200 mg daily. For example, palbociclib has been approved at a dose of 125 mg once daily. Palbociclib has also been approved at a reduced dose such as 75 mg once daily and 100 mg once daily. In embodiments, ribociclib may be administered from 50 mg to 800 mg daily. For example, ribociclib has been approved at a dose of 600 mg once daily. Ribociclib has also been approved at a reduced dose such as 400 mg once daily and 200 mg once daily. In embodiments, abemaciclib may be administered from 100 mg to 800 mg daily. For example, ribociclib has been approved at a dose of 150 mg twice daily and 200 mg twice daily. Ribociclib has also been approved at a reduced dose such as 100 mg twice daily and 50 mg twice daily. It will be appreciated that each of the recited ranges above can include any sub-range or sub-point therein, inclusive of endpoints. It will be appreciated that each of the recited ranges above can include any sub-range or sub-point therein, inclusive of endpoints. Tablets or other forms of presentation provided in discrete units may conveniently contain an amount of one or more compounds which is effective at such dosage or as a multiple of the same, for instance, units containing 5 mg to 500 mg, usually around 10 mg to 200 mg. The amount of active ingredient that may be combined with the carrier materials to produce a single dosage form will vary depending upon the host treated and the particular mode of administration. In embodiments, the administration is oral.

In embodiments, the methods disclosed herein can be coupled with diagnostics. For example, the methods disclosed here can be combined with a step of selecting a patient or subject. Such a selecting step may be based on a biomarker such as a KRAS mutation, or a mutation in the CDK4/6 pathway. The efficacy of this combination can be observed in specific tumor types, such as non-small cell lung cancer (NSCLC) and colorectal cancer (CRC). In embodiments, there are provided methods of treating colorectal cancer in a subject comprising orally administering to the subject a therapeutically effective amount of a compound of Formula I or its pharmaceutically acceptable salt in combination with a CDK4/6 inhibitor. In embodiments, there are provided methods of treating NSCLC in a subject comprising orally administering to the subject a therapeutically effective amount of a compound of Formula I or its pharmaceutically acceptable salt in combination with a CDK4/6 inhibitor. In embodiments, the compound of Formula I is administered once or twice daily. In embodiments, CDK4/6 inhibitor is administered once or twice daily. The drugs can be co-administered as described herein, for example.

In embodiments, the subject is a human. In embodiments, the subject is a mammal other than a human, such as a primate, a rodent, a dog, a cat, or other small animal.

In embodiments, there are provided methods of inhibiting growth of cancer cells, comprising contacting a cancer cell population with Formula I or its pharmaceutically acceptable salt in combination with a CDK4/6 inhibitor. In some embodiments, a concentration of the compound of Formula I is in a range from 1 nM to 1,000 nM. In some embodiments, a concentration of the compound of Formula I is in a range from 1 nM to 500 nM. In some embodiments, a concentration of the compound of Formula I is in a range from 1 nM to 100 nM. In some embodiments, a concentration of the compound of Formula I is in a range from 1 nM to 20 nM. In some embodiments, a concentration of the compound of Formula I is in a range from 1 nM to 10 nM.

In some embodiments, a concentration of CDK4/6 inhibitor is in a range from 10 nM to 1000 nM. In some embodiments, a concentration of CDK4/6 inhibitor is in a range from 10 nM to 500 nM. In some embodiments, a concentration of CDK4/6 inhibitor is in a range from 10 nM to 250 nM. In some embodiments, a concentration of CDK4/6 inhibitor is in a range from 10 nM to 100 nM. In some embodiments, a concentration of CDK4/6 inhibitor is in a range from 10 nM to 50 nM.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the cancer has a class 1 B-Raf mutation.

In some embodiments, the cancer harbors at least one of a EGFR, KRAS, BRAF (e.g., BRAF class III) and/or NF1 (e.g., loss of function) mutations.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12R KRAS mutation. In some embodiments, the cancer has a G12S KRAS mutation. In some embodiments, the cancer has a G12V KRAS mutation. In some embodiments, the cancer has G12W KRAS mutation. In some embodiments, the cancer has a G13D KRAS mutation. In some embodiments, the cancer has a H95D KRAS mutation. In some embodiments, the cancer has a H95Q KRAS mutation. In some embodiments, the cancer has a H95R KRAS mutation. In some embodiments, the cancer has a Q16H KRAS mutation. In some embodiments, the cancer has a Q61H KRAS mutation. In some embodiments, the cancer has a Q16K KRAS mutation. In some embodiments, the cancer has a Q61R NRAS mutation. In some embodiments, the cancer has a R68S KRAS mutation.

In some embodiments, the cancer is a MAPKm/MAPKi-naïve pancreatic cancer. In some embodiments, the pancreatic cancer is pancreatic ductal adenocarcinoma (PDAC).

In some embodiments, the cancer comprises one or more EGFR mutation selected from the group consisting of EGFR gene copy gain, EGFR gene amplification, chromosome 7 polysomy, L858R, exon 19 deletions/insertions, L718Q, L861Q, G719C, G719S, G724S, G719A, V765A, T783A, exon 20 insertions, EGFR splice variants (Viii, Vvi, and Vii), A289D, A289T, A289V, G598A, G598V, T790M, S768I, C797X, and C797S. In some embodiments, the cancer comprises one or more EGFR mutation selected from the group consisting of L858R, exon 19 deletion, and T790M.

In some embodiments, the cancer is the cancer is a liquid tumor. In some embodiments, the cancer is the liquid tumor is leukemia. In some embodiments, the cancer is the leukemia is acute myeloid leukemia (AML). In some embodiments, the cancer is the AML is relapsed and/or refractory AML. In some embodiments, the cancer is the AML is a FLT3 mutant AML.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer, gallbladder cancer, liver cancer, pancreatic cancer, small intestine cancer, or stomach cancer (gastric cancer).

In some embodiments, the cancer is non-small cell lung cancer (NSCLC). In some embodiments, the NSCLC is an EGFR mutant NSCLC. In some embodiments, the NSCLC is a KRAS^{G12C} mutant NSCLC. In some embodiments, the NSCLC is a KRAS^{G12D} mutant NSCLC. In some embodiments, the NSCLC is a KRAS^{G12S} mutant NSCLC. In some embodiments, the NSCLC is a KRAS^{G12V} mutant NSCLC. In some embodiments, the NSCLC is a KRAS^{G13D} mutant NSCLC. In some embodiments, the NSCLC is a KRAS^{Q61H} mutant NSCLC. In some embodiments, the NSCLC is a KRAS^{Q61K} mutant NSCLC. In some embodiments, the EGFR mutation is an acquired EGFR mutation. In some embodiments, the acquired EGFR mutation is C797X. In some embodiments, the acquired EGFR mutation is L718Q. In some embodiments, the acquired EGFR mutation is EGFR gene amplification. In some embodiments, the acquired EGFR mutation is G724S. In some embodiments, the acquired EGFR mutation is S768I.

In some embodiments, the NSCLC is a NRAS^{Q61R} mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC. In some embodiments, the cancer is a KRAS-treated G12R NSCLC. In some embodiments, the cancer is a KRAS-treated G12W NSCLC. In some embodiments, the cancer is a KRAS-treated H95D NSCLC. In some embodiments, the cancer is a KRAS-treated H95Q NSCLC. In some embodiments, the cancer is a KRAS-treated H95R NSCLC. In some embodiments, the cancer is a KRAS-treated G12D NSCLC. In some embodiments, the cancer is a KRAS-treated R68S NSCLC.

In some embodiments, the cancer is pancreatic cancer. In some embodiments, the cancer is a MAPKm/MAPKi-naïve pancreatic cancer. In some embodiments, the pancreatic cancer is pancreatic ductal adenocarcinoma (PDAC).

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12R mutant CRC. In some embodiments, the CRC is a KRAS G12S mutant CRC. In some embodiments, the CRC is a KRAS G12V mutant CRC. In some embodiments, the CRC is a KRAS G12W mutant CRC. In some embodiments, the CRC is a KRAS G13D mutant CRC. In some embodiments, the CRC has a H95D KRAS mutation. In some embodiments, the CRC has a H95Q KRAS mutation. In some embodiments, the CRC has a H95R KRAS mutation. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC. In some embodiments, the CRC has a R68S KRAS mutation.

In some embodiments, the cancer is esophageal cancer.

In some embodiments, the cancer has one or more acquired mutations. In some embodiments, the acquired mutation results from a first-line treatment. In some embodiments, the first-line treatment is an EGFR inhibitor. In some embodiments, the EGFR inhibitor is osimertinib. In some embodiments, the first-line treatment is a KRAS inhibitor. In some embodiments, the KRAS inhibitor is a KRAS G12C inhibitor. In some embodiments, the KRAS G12C inhibitor is adagrasib. In some embodiments, the KRAS G12C inhibitor is sotorasib. In some embodiments, the cancer is a solid tumor cancer. In some embodiments, the cancer is NSCLC.

In some embodiments, the acquired mutation is an acquired EGFR mutation. In some embodiments, the acquired EGFR mutation is C797X. In some embodiments, the acquired EGFR mutation is L718Q. In some embodiments, the acquired EGFR mutation is EGFR amplification. In some embodiments, the acquired EGFR mutation is G724S. In some embodiments, the acquired mutation is S768I.

In some embodiments, the acquired mutation is an acquired amplification mutation. In some embodiments, the acquired mutation is a MET gene amplification. In some embodiments, the acquired mutation is HER2 gene amplification.

In some embodiments, the acquired mutation is an acquired oncogenic fusion. In some embodiments, the acquired oncogenic fusion is SPTBN1-ALK. In some embodiments, the acquired oncogenic fusion is RET fusion. In some embodiments, the acquired oncogenic fusion is BRAF fusion. In some embodiments, the acquired mutation is an acquired MAPK-PI3K mutation. In some embodiments, the acquired MAPK-PI3K mutation is BRAF-V600E. In some embodiments, the acquired MAPK-PI3K mutation is PI3KCA. In some embodiments, the acquired MAPK-PI3K mutation is KRAS. In some embodiments, the acquired MAPK-PI3K mutation is HER2.

In some embodiments, the acquired mutation is an acquired KRAS mutation. In some embodiments, the acquired mutation is KRASG^{12C}. In some embodiments, the acquired mutation is KRAS G12D. In some embodiments, the acquired mutation is KRAS^{G12R}. In some embodiments, the acquired mutation is KRAS^{G12V}. In some embodiments, the acquired mutation is KRAS^{G12W}. In some embodiments, the acquired mutation is KRAS^{G13D}. In some embodiments, the acquired mutation is KRAS^{H95D}. In some embodiments, the acquired mutation is KRAS^{H95Q}. In some embodiments, the acquired mutation is KRAS^{H95R}. In some embodiments, the acquired mutation is KRAS^{Q61H}. In some embodiments, the acquired mutation is KRAS^{R68S}.

In some embodiments, the acquired mutation is an acquired MAPK pathway mutation. In some embodiments, the acquired MAPK pathway mutation is MAP2K1 K57N. In some embodiments, the acquired MAPK pathway mutation is MAP2K1 K57T. In some embodiments, the acquired MAPK pathway mutation is CCDC6-RET. In some embodiments, the acquired MAPK pathway mutation is RITI P128L. In some embodiments, the acquired MAPK pathway mutation is PTEN G209V. In some embodiments, the acquired MAPK pathway mutation is BRAF V600E. In some embodiments, the acquired MAPK pathway mutation is MAP2K1 199_K104del. In some embodiments, the acquired MAPK pathway mutation is MAP2K1 K57N. In some embodiments, the acquired MAPK pathway mutation is EML4-ALK. In some embodiments, the acquired MAPK pathway mutation is EGFR A289A. In some embodiments, the acquired MAPK pathway mutation is FGFR3-TACC3. In some embodiments, the acquired MAPK pathway mutation is AKAP9-BRAF. In some embodiments, the acquired MAPK pathway mutation is RAF1-CCDC176. In some embodiments, the acquired MAPK pathway mutation is RAF1-TRAK1. In some embodiments, the acquired MAPK pathway mutation is NRAS Q61K. In some embodiments, the acquired MAPK pathway mutation is MAP2K1 E102_1103DEL. In some embodiments, the acquired MAPK pathway mutation is NRF1-BRAF.

In some embodiments, the acquired mutation is a KRAS G12C reactivation mutation. In some embodiments, the KRAS G12C reactivation mutation is a RKRAS G12C gene amplification. In some embodiments, the KRAS G12C reactivation mutation is a NF1 R22637 (LoF).

In some embodiments, the acquired mutation is a non-G12C activation KRAS mutation. In some embodiments, the non-G12C activation KRAS mutation is KRAS G12D. In some embodiments, the non-G12C activation KRAS mutation is KRAS G12R. In some embodiments, the non-G12C activation KRAS mutation is KRAS G12V. In some embodiments, the non-G12C activation KRAS mutation is KRAS G12W. In some embodiments, the non-G12C activation KRAS mutation is KRAS G13D. In some embodiments, the non-G12C activation KRAS mutation is KRAS Q61H. In some embodiments, the non-G12C activation KRAS mutation is KRAS Q61K.

In some embodiments, the acquired mutation is a sterically hindering KRAS G12C mutation. In some embodiments, the sterically hindering KRAS G12C mutation is KRAS R68S. In some embodiments, the sterically hindering KRAS G12C mutation is KRAS H95D. In some embodiments, the sterically hindering KRAS G12C mutation is KRAS H95Q. In some embodiments, the sterically hindering KRAS G12C mutation is KRAS H95R. In some embodiments, the sterically hindering KRAS G12C mutation is KRAS Y96C.

In some embodiments, the acquired mutation is an RTK activation mutation. In some embodiments, the RTK activation mutation is EGFR A289V. In some embodiments, the RTK activation mutation is RET M918T. In some embodiments, the RTK activation mutation is MET gene amplification. In some embodiments, the RTK activation mutation is EML-ALK. In some embodiments, the RTK activation mutation is CCDC6-RET. In some embodiments, the RTK activation mutation is FGFR3-TACC3.

In some embodiments, the acquired mutation is a downstream RAS/MAPK activation mutation. In some embodiments, the downstream RAS/MAPK activation mutation is BRAF V600E. In some embodiments, the downstream RAS/MAPK activation mutation is MAP2K I99_K104del. In some embodiments, the downstream RAS/MAPK activation mutation is MAP2K1 I99_K104del. In some embodiments, the downstream RAS/MAPK activation mutation is MAP2K1 E102_I103del. In some embodiments, the downstream RAS/MAPK activation mutation is RAF fusion.

In some embodiments, the acquired mutation is a parallel pathway activation mutation. In some embodiments, the parallel pathway activation mutation is PIK3CA H1047R. In some embodiments, the parallel pathway activation mutation is PIK3R1 S361fs. In some embodiments, the parallel pathway activation mutation is PTEN N48K. In some embodiments, the parallel pathway activation mutation is PTEN G209V. In some embodiments, the parallel pathway activation mutation is RIT1 P128L.

### Compositions

The compound of Formula I disclosed herein may exist as salts. The present embodiments includes such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present embodiments contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference n its entirety for all of its teachings, including without limitation all methods, compounds, compositions, data, and the like, for use with any of the embodiments and disclosure herein..

Pharmaceutically acceptable salts includes salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data, and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present embodiments. The compounds of the present embodiments do not include those that are known in art to be too unstable to synthesize and/or isolate. The present embodiments is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

In some embodiments, there are provided pharmaceutical compositions comprising the compound of Formula I and a pharmaceutically acceptable excipient. In some embodiments, the pharmaceutical compositions are configured as an oral tablet preparation.

The compounds of the present embodiments can be prepared and administered in a wide variety of oral, parenteral, and topical dosage forms. Oral preparations include tablets, pills, powder, dragees, capsules, liquids, lozenges, gels, syrups, slurries, suspensions, etc., suitable for ingestion by the patient. The compounds of the present embodiments can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. The compounds of formula I disclosed herein can also be administered by in intraocular, intravaginal, and intrarectal routes including suppositories, insufflation, powders, and aerosol formulations (for examples of steroid inhalants, see Rohatagi, J. Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data, and the like, for use with any of the embodiments and disclosure herein. Accordingly, the present embodiments also provides pharmaceutical compositions including one or more pharmaceutically acceptable carriers and/or excipients and either a compound of formula I, or a pharmaceutically acceptable salt of a compound of formula I.

For preparing pharmaceutical compositions from the compounds of the present embodiments, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data, and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating, or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain the compounds of formula I mixed with a filler or binders such as lactose or starches, lubricants such as talc or magnesium stearate, and, optionally, stabilizers. In soft capsules, the compounds of formula I may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame, or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data, and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of the compound of Formula I disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J Pharm. Sci. 84:1144-1146; Rohatagi (1995) Pharmazie 50:610-613; Brophy (1983) Eur. J Clin. Pharmacol. 24:103-108; the latest Remington's, supra; each of which is incorporated herein by reference in its entirety for all of its teachings, including without limitation all methods, compounds, compositions, data, and the like, for use with any of the embodiments and disclosure herein.). The state of the art allows the clinician to determine the dosage regimen for each individual patient, GR and/or MR modulator and disease or condition treated.

Single or multiple administrations of the compound of Formula I formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compound of formula I is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of formula I for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987), which is incorporated herein by reference in its entirety for all of its teachings, including without limitation all methods, compounds, compositions, data and the like, for use with any of the embodiments and disclosure herein.

In some embodiments, co-administration includes administering one active agent within 0.5, 1, 2, 4, 6, 8, 10, 12, 16, 20, or 24 hours (or any sub-range of time or sub-value of time within a 24 hour period) of a second active agent. Co-administration includes administering two active agents simultaneously, approximately simultaneously (e.g., within about 1, 5, 10, 15, 20, or 30 minutes of each other (or any sub-range of time or sub-value of time from 0-30 minutes for example)), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another. At least one administered dose of drugs can be administered, for example, at the same time. At least one administered dose of the drugs can be administered, for example, within minutes or less than an hour of each other. At least one administered dose of drugs can be administered, for example, at different times, but on the same day, or on different days.

After a pharmaceutical composition including a compound of formula I disclosed herein has been formulated in one or more acceptable carriers, it can be placed in an appropriate container and labeled for treatment of an indicated condition. For administration of the compounds of formula I, such labeling would include, e.g., instructions concerning the amount, frequency, and method of administration.

### Kits and Products

Some embodiments relate to kits and products that include the compound of Formula 1 and/or at least one CDK4/6 inhibitor. For example, the kit or product can include a package or container with a compound of Formula I. Such kits and products can further include a product insert or label with approved drug administration and indication information, including how to use the compound of Formula 1 in combination with a CDK4/6 inhibitor that is separately provided. The kits can be used in the methods of treating cancer as described herein.

In some aspects, the kits or products can include both a compound of Formula 1 and at least one CDK4/6 inhibitor. In some embodiments, the CDK4/6 inhibitor is palbociclib, ribociclib, abemaciclib, FCN-437c, or alvociclib. Such kits can include one or more containers or packages, which include one or both combination drugs together in a single container and/or package, or in separate packages/containers. In some instances, the two drugs are separately wrapped, but included in a single package, container, or box. Such kits and products can further include a product insert or label with approved drug administration and indication information, including how to use the compound of Formula 1 in combination with a CDK4/6 inhibitor. The kits can be used in the methods of treating cancer as described herein.

### EXAMPLES

### General Procedures

All starting materials and solvents were obtained either from commercial sources or prepared according to the literature citation.

### Example 1: Combination of the Compound of Formula I with Palbociclib Showed a Robust Inhibition of Colony Formation in KRAS^{G12D}, KRAS^{G12V} and KRAS^{G12S} Mutated NSCLC and CRC Cells

This study was conducted to determine the synergistic effect of combining Formula I with a CDK4/6 inhibitor. The combination of Formula I with palbociclib showed a robust inhibition of colony formation, for example in cancer cell lines harboring oncogenic KRAS^{G12D}, KRAS^{G12S}, and KRAS^{G12V} mutations.

Cells and Reagents: The cell lines were obtained from ATCC (SW620 #CCL-227, A549 #CCL-185 and NCI-H441 #HTB-174). The cell lines, LS180 (ECACC #87021202) and Gp2D (ECACC #95090714). The cell line, CAL 27 was cultured in DMEM (Gibco #). SCC-4, SCC-15, SCC-25, SCC-9 cell lines were cultured in a 1:1 mixture of DMEM and Ham's F12 medium containing 1.2 g/L sodium bicarbonate, 2.5 mM L-glutamine, 15 mM HEPES and 0.5 mM sodium pyruvate supplemented with 400 ng/ml hydrocortisone, 90%; fetal bovine serum, 10% (Hyclone #SH-30071.03) and Penicillin/Streptomycin (Thermo Fisher #15070-063). The cells were maintained at 37° C./5% CO₂.

Clonogenic assay: The cells were plated onto 6-well plates in 2 ml cell culture medium. The cells were incubated overnight and treated with various concentration of the compound of Formula I and Palbociclib. After 7 days incubation, the medium was replaced with fresh medium, cells were treated again with same concentration of the compound of Formula I and Palbociclib and incubated for additional 7 days. After 14 days of total incubation, the cells were washed with PBS twice and fixed 30 mins with 4% formaldehyde. The cells were washed twice with PBS and incubated with 0.1% crystal violet for 60 mins. After the crystal violet staining, the cells were washed five times with water and let it dry at room temperature. After the plates were dried, the crystal staining was de-stained with 1 ml of 10% acetic acid and absorbance was measured at 560 nM.

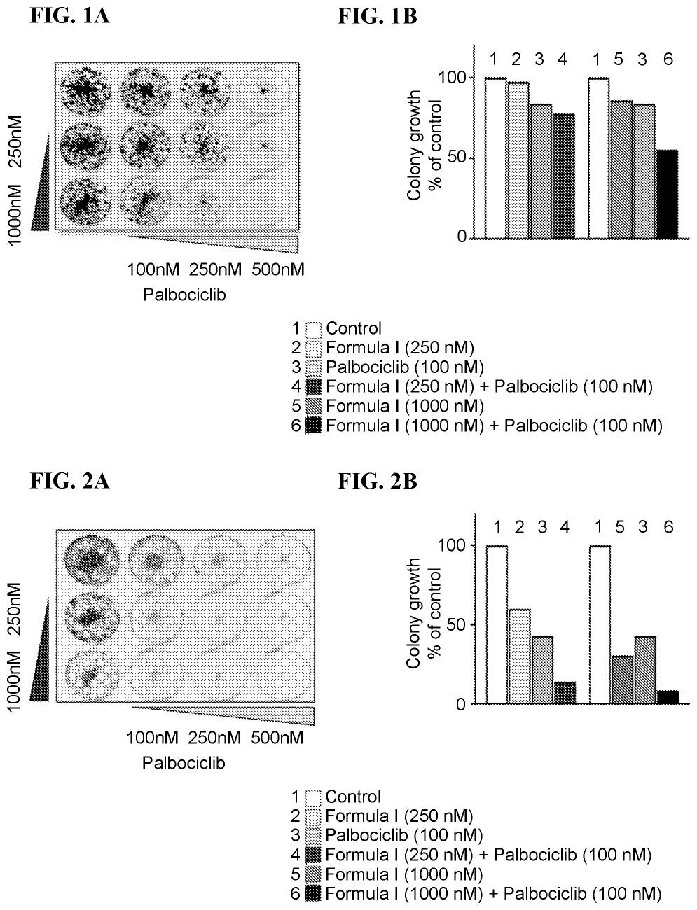

The results of these experiments are indicated in FIGS. 1A to 5B and are further discussed below.

### Results

Cell lines from each of KRAS^{G12D} (LS513 and Gp2D), KRAS^{G12S} (A549) and KRAS^{G12V} (NCI-H441 and SW620) mutations were split onto 6-well plates in 2 ml cell culture medium. The cells were incubated overnight and treated with various concentration of the compound of Formula I and Palbociclib and assayed using the Clonogenic Assay protocol described above.

The A549 cells were split onto 6 well plates (2000 cells per well). After overnight incubation, the cells were treated with the compound of Formula I alone and treated in combination with palbociclib and colony formation was determined as mentioned in method. The compound of Formula I inhibited the colony formation minimally. The Palbociclib treatment showed inhibition of colony formation dose dependently. The combination of the compound of Formula I and Palbociclib showed a robust reduction of colony formation. (FIG. 1A). The quantification of colony formation data showed that treatment with the compound of Formula I and palbociclib alone reduced the colony formation about 10% and combination of the compound of Formula I and palbociclib showed a pronounced (about 40%) reduction of colony formation (FIG. 1B).

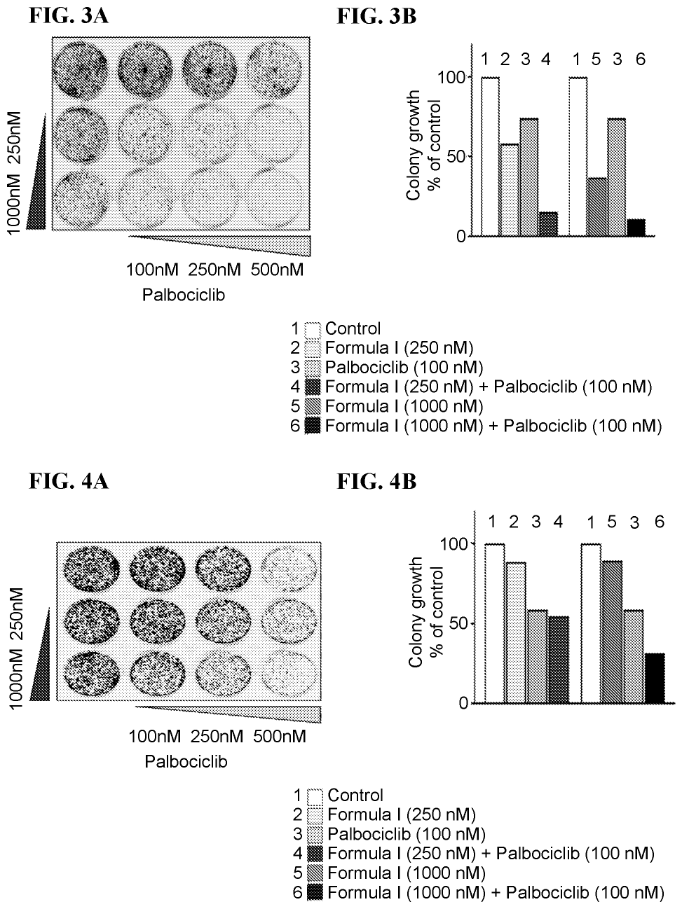

The NCI-H441 and SW620 cells were split onto 6 well plates (8000 and 4000 calls per well respectively). After overnight incubation, the cells were treated with the compound of Formula I alone and treated in combination with palbociclib and colony formation was determined as mentioned in method. The compound of Formula I inhibited the colony formation minimally. The Palbociclib treatment showed inhibition of colony formation dose dependently. The combination of the compound of Formula I and Palbociclib showed a robust reduction of colony formation. (FIGS. 2A&3A). In NCI-H441, the quantification of colony formation data showed that treatment with the compound of Formula I (250 nM) showed about 40% of colony formation. The palbociclib (100 nM) treatment inhibited the colony formation about 60%. There was a pronounced inhibition of colony formation (about 80%) observed with combination of the compound of Formula I and palbociclib treatment. Increasing the concentration of the compound of Formula I showed a robust inhibition of colony formation and suggesting a dose dependent effect on colony formation (FIG. 2B). In SW620, the quantification of colony formation data showed that treatment with the compound of Formula I (250 nM) showed about 10% of colony formation. The palbociclib (100 nM) treatment inhibited the colony formation about 40%. There was a pronounced inhibition of colony formation (about 60%) observed with combination of the compound of Formula I and palbociclib treatment. Increasing the concentration of the compound of Formula I showed robust inhibition of colony formation by single agent and combination treatment (FIG. 3B).

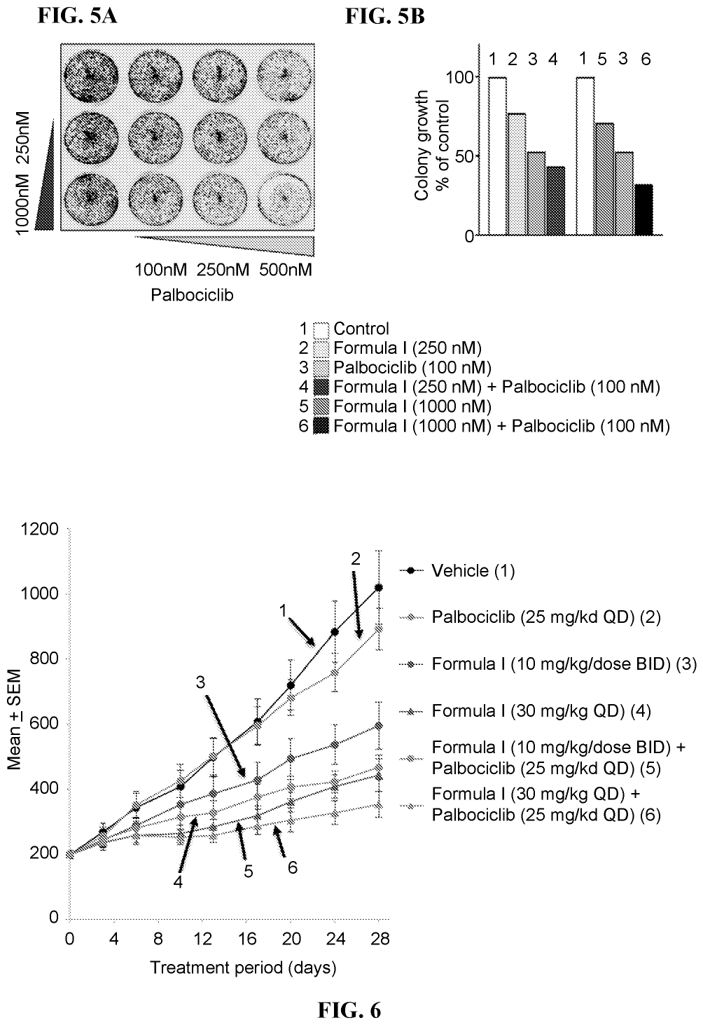

The LS-180 and Gp2D cells were split onto 6 well plates (4000 and 8000 calls per well respectively). After overnight incubation, the cells were treated with the compound of Formula I alone and treated in combination with palbociclib and colony formation was determined as mentioned in method. The compound of Formula I inhibited the colony formation minimally. The Palbociclib treatment showed inhibition of colony formation dose dependently. The combination of the compound of Formula I and Palbociclib showed a robust reduction of colony formation. (FIGS. 4A&5A). In LS-180, the quantification of colony formation data showed that treatment of the compound of Formula I (250 nM) showed about 10% of colony formation. The palbociclib (250 nM) treatment inhibited the colony formation about 40%. There was a pronounced inhibition of colony formation (about 50%) observed with combination of the compound of Formula I and palbocilib treatment. Increasing the concentration of the compound of Formula I (1000 nM) showed a pronounced inhibition of colony formation about 60% when combined with palbociclib (FIG. 4B). In Gp2D, the quantification of colony formation data showed that treatment of the compound of Formula I (250 nM) showed about 20% of colony formation. The palbociclib (250 nM) treatment inhibited the colony formation about 40%. There was a pronounced inhibition of colony formation (about 50%) observed with combination of the compound of Formula I and palbocilib treatment. Increasing the concentration of the compound of Formula I (1000 nM) showed a pronounced inhibition of colony formation about 70% when combined with palbociclib (FIG. 5B).

### Example 2: Combination Efficacy of the Compound of Formula I and Palbociclib in the KRASG12S Mutant NSCLC Xenograft Model A549

Reagents: The vehicle/control article, 100 mM acetic acid in deionized water, with pH adjustment to 4.8-5.0, was prepared and stored under ambient conditions throughout the 28-day administration in mice. The test article compound of Formula I was freshly prepared in vehicle of 100 mM acetic buffer weekly and stored under ambient conditions. The combination agent palbociclib was freshly prepared in vehicle of 50 mM Sodium Lactate Buffer, pH 3.8-4.0 weekly and stored at 2-8° C.

Animals: Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

Preparation of xenograft model: A549 was a human lung cancer cell line that harbored a KRASG12S mutation. The A549 cell line was purchased from the American Type Culture Collection (ATCC® CCL-185™). A549 cells were cultured in medium containing RPMI1640 plus 10% Fetal Bovine Serum (FBS) at 37° C. in an atmosphere of 5% CO2 in air. The medium was renewed every 2 to 3 days and tumor cells were routinely sub-cultured at a confluence of 80-90% by trypsin-EDTA. The cells growing in an exponential growth phase were harvested and counted for inoculation.

A549 tumor cells were implanted into mice subcutaneously. Briefly, 200 μL cell suspensions containing 5×10⁶ tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 200 mm³ (range of 152-259 mm³), tumor-bearing mice were randomized into different groups with 8 mice in each group. The randomization date was denoted as treatment day 0.

Treatment: Treatment started on the day of randomization. The treatment start day was denoted as treatment day 0. Mice were dosed by oral administration of vehicle control solution, the compound of Formula I at 10 mg/kg/dose BID, the compound of Formula I at 30 mg/kg QD, or palbociclib at 25 mg/kg QD monotherapy treatment groups. Two additional groups received combination treatment of the compound of Formula I and palbociclib, one dosing the compound of Formula I at 10 mg/kg/dose BID with palbociclib at 25 mg/kg QD and the other dosing the compound of Formula I at 30 mg/kg QD with palbociclib at 25 mg/kg QD. The dosing volume for each compound was 5 mL/kg and interval of BID regimen was 8 hours. Palbociclib was dosed at one-hour post the compound of Formula I QD or first BID dose in combination groups. In addition to regular food and water supply, DietGel (ClearH2O, US) was added in cages where one or more mice showed >10% BWL. Per this practice, mice in the compound of Formula I at 10 mg/kg/dose BID monotherapy group and in the compound of Formula I at 10 mg/kg/dose BID with palbociclib at 25 mg/kg QD combination group were supplied with DietGel food starting on treatment day 20 and continuing through the remaining study period. Mice in the compound of Formula I at 30 mg/kg QD monotherapy group were supplied with DietGel food starting on treatment day 17 and continuing through the remaining study period. The study was terminated on treatment day 28 as being defined in the study protocol.

Measurements and calculations: Conditions of animal health and clinical signs of toxicity were monitored daily. Subcutaneous tumor volumes and mouse body weights were measured twice a week by caliper and balance, respectively. Tumor volume, percent of tumor growth inhibition (% TGI), percent of tumor regression, percent of body weight change (% BWC), and treatment related body weight loss (% trBWL) were calculated based on following formulas:

$$Tumor volume ( TV ) = ( length \times width 2 ) / 2$$

$$Percentage of body weight change at treatment day \times ( \% BWCdx ) =$$

$$[ ( BWdx - BWd 0 ) / WBd 0 ] \times 100$$

i. BWdx was body weight group mean at treatment day x
ii. BWd0 was body weight group mean at treatment day 0

Percentage of treatment related body weight loss at treatment day x (% trBWLdx) was calculated based on the following:
iii. If % BWC in the treatment group was positive at treatment day x: % trBWLdx=0%
iv. If % BWC was negative in both the control and treatment groups at treatment day
   x:

$$\% trBWLdx = \% BWCCdx - \% BWCTdx$$

% BWCCdx was the % BWC group mean observed in the control group at day x

% BWCTdx was the % BWC group mean observed in the treatment group at day x
v. If positive % BWC was observed in the control group and negative % BWC was observed in the treatment group at treatment day x: % trBWLTdx=% BWCTdx x−1
vi. Summary treatment related body weight loss (% trBWL) per treatment group was calculated by taking the maximum trBWLdx % observed during the study.

Percent of tumor growth inhibition (% TGI)
vii.

$$TGI \% = [ 1 - ( TVTi - TVT 0 ) / ( TVCi - TVC 0 ) ] \times 100$$

viii. TVTi was the group mean tumor volume (TV) of treatment group at last treatment day
ix. TVT0 was the group mean TV of treatment group at treatment day 0
x. TVCi was the group mean TV of control group at last treatment day
xi. TVC0 was the group mean TV of control group at treatment day 0

Percent of tumor regression (% Regression)
xii.

$$\% Regression = 100 \times ( TV 0 - TVi ) / TV 0$$

xiii. TV0 was the group mean TV in the same group but measured at the treatment day 0
xiv. TVi was the group mean TV in the same group but measured at the last treatment day

Statistical analysis: Graphed data points represented group means and error bars represented SEM. P-values based on the comparison of two groups were calculated by the unequal variances t-test (two-tailed). Tumor volume measurements from the last day of treatment were used to calculate significance. Accounting for multiple hypothesis testing, p-values were adjusted using the Benjamini-Hochberg Method. Adjusted p-values <0.05 were defined as statistically significant.

Treatment related body weight loss (trBWL) was categorized based on the following criteria: x≤5% was no loss, 5%<x≤10% was mild loss, 10%<x≤20% was moderate loss and x>20% was severe loss.

### Results and Discussion

Antitumor efficacy: The compound of Formula I statistically significantly inhibited tumor growth as a monotherapy and in combination with a CDK4/6 inhibitor, palbociclib, in the KRAS^{G12S} mutant NSCLC xenograft model A549. Compared to vehicle control, 28-day repeated oral administration of the compound of Formula I at 10 mg/kg/dose BID and the compound of Formula I at 30 mg/kg QD monotherapy treatment inhibited tumor growth by 52% (adjusted p-value <0.05) and by 70% (adjusted p-value <0.05), respectively. Repeated oral administration of palbociclib at 25 mg/kg QD inhibited tumor growth by 15% (adjusted p-value ≥0.05) (FIG. 6 and Table 1). Combinations of the compound of Formula I at 10 mg/kg/dose BID with palbociclib at 25 mg/kg QD and the compound of Formula I at 30 mg/kg QD with palbociclib at 25 mg/kg QD statistically significantly inhibited tumor growth by 67 (adjusted p-value t 0.05) and 81 (adjusted p-value <0.05), respectively (FIG. 6 and Table 1). Observed TGIs in the compound of Formula I at 10 mg/kg/dose BID or the compound of Formula I at 30 mg/kg QD with palbociclib at 25 mg/kg QD combination treatment groups were statistically significant relative to the palbociclib monotherapy treatment group (adjusted p-values <0.05) but were not statistically significant relative to the respective compound of Formula I monotherapy groups (adjusted p-values >0.05) (FIG. 6 and Table 2).

TABLE 1
Summary of the efficacy of the compound of Formula
I in the KRAS^{G12S} mutant NSCLC xenograft model A549

| | | | | | | Unequal | |
|---|---|---|---|---|---|---|---|
| | | | | | % Tumor | variances | |
| | Dose | | Number of | % TGI | regression | t-test p- | Adjusted |
| | level | | mice at last | (last | (last | value | p-value |
| Treatment | (mg/ | | treatment | treatment | treatment | (vs. | (vs. |
| group | kg) | Regimen | day | day) | day) | vehicle) | vehicle) |
| Vehicle | 0 | BID | 8 | — | — | — | — |
| Palbociclib | 25 | QD | 8 | 15 | 0 | 3.4E−01 | 3.4E−01 |
| Formula I | 10 | BID | 8 | 52 | 0 | 8.3E−03 | 1.2E−02 |
| Formula I | 30 | QD | 8 | 70 | 0 | 9.8E−04 | 2.2E−03 |
| Formula I + | 10 | BID | 8 | 67 | 0 | 1.4E−03 | 2.5E−03 |
| Palbociclib | 25 | QD | | | | | |
| Formula I + | 30 | QD | 8 | 81 | 0 | 3.9E−04 | 1.1E−03 |
| Palbociclib | 25 | QD | | | | | |

TABLE 2
Summary of the combination efficacy of Formula I relative to palbociclib and
Formula I monotherapies in the KRAS^{G12S} mutant NSCLC xenograft model A549

| | | | Unequal | | | |
|---|---|---|---|---|---|---|
| | | | variances | Adjusted | Unequal | |
| | Dose | | t-test p-value | p-value (vs. | variances | Adjusted |
| | level | | (vs. respective | respective | t-test p-value | p-value |
| Treatment | (mg/ | | palbociclib | palbociclib | (vs. Formula I | (vs. Formula I |
| group | kg) | Regimen | monotherapy) | monotherapy) | monotherapy) | monotherapy) |
| Formula I + | 10 | BID | 1.1E−04 | 5.1E−04 | 1.4E−01 | 1.8E−01 |
| Palbociclib | 25 | QD | | | | |
| Formula I + | 30 | QD | 1.2E−05 | 1.1E−04 | 1.8E−01 | 2.0E−01 |
| Palbociclib | 25 | QD | | | | |

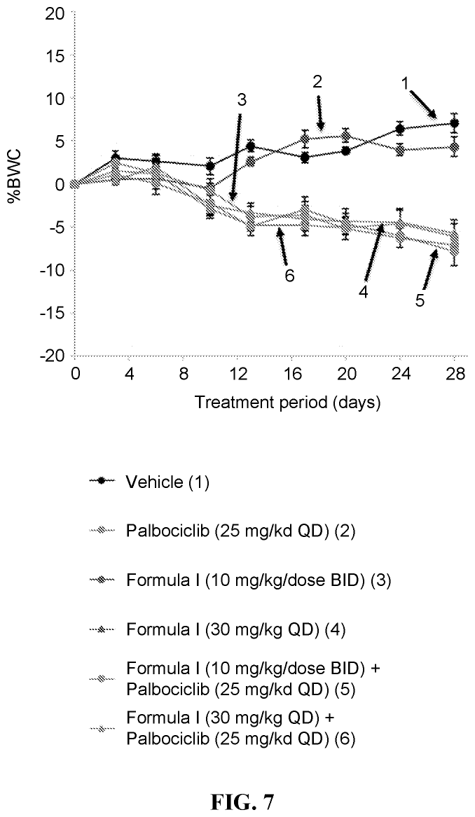

Body weight change: There was no body weight loss in mice treated with vehicle solution. Mice in the palbociclib at 25 mg/kg QD monotherapy treatment group had no trBWL (400 maximum trBWL). Mice in both the compound of Formula I monotherapy treatment groups and both combination treatment groups had mild trBWL (≤8% maximum trBWL) during the study period of 28 days (FIG. 7). In addition to regular food and water supply, DietGel was added in cages where one or more mice showed >10% BWL. Per this practice, mice in the compound of Formula I at 10 mg/kg/dose BID monotherapy group and the compound of Formula I at 10 mg/kg/dose BID with palbociclib at 25 mg/kg QD combination group were supplied with DietGel food starting on treatment day 20 and continuing through the remaining study period. Mice in the compound of Formula I at 30 mg/kg QD monotherapy group were supplied with DietGel food starting on treatment day 17 and continuing through the remaining study period. In addition to mild trBWL in both the compound of Formula I monotherapy treatment groups and both combination treatment groups, no clinical signs of toxicity or mortality were observed during the treatment period in this study.

Conclusion: The compound of Formula I statistically significantly inhibited tumor growth as a monotherapy and in combination with a CDK4/6 inhibitor, palbociclib, in the KRAS^{G12S} mutant NSCLC xenograft model A549. Combination treatment groups of the compound of Formula I at 10 mg/kg/dose BID or the compound of Formula I at 30 mg/kg QD with palbociclib at 25 mg/kg QD were statistically significant relative to the palbociclib monotherapy treatment group (adjusted p-values <0.05) but were not statistically significant relative to the respective the compound of Formula I monotherapy groups. In addition to mild trBWL in both the compound of Formula I monotherapy treatment groups and both combination treatment groups, no clinical signs of toxicity or mortality were observed during the treatment period in this study.

### Example 3. Combination Efficacy of the Compound of Formula I and Palbociclib in KRASG13D NSCLC CDX A-427

### Vehicle/Control Article

The vehicle/control article of Formula I, 100 mM acetic acid in deionized water, with pH adjustment to 4.8-5.0, was prepared and stored under ambient conditions throughout the 28-day administration in mice.

The vehicle/control article of palbociclib, 50 mM sodium lactate in deionized water, with pH adjustment to 3.8-4.0, was prepared and stored at 2-8° C. throughout the 28-day administration in mice.

### Formulation of Test Article

The test article Formula I was freshly prepared in vehicle of 50 mM acetic buffer weekly and stored under ambient conditions. The combination agent palbociclib was prepared weekly in vehicle of 50 mM sodium lactate buffer, pH 3.8-4.0, and stored at 2-8° C.

### Animals

Female SCID Beige mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

### Preparation of Xenograft Model

The parental cell line, A-427, was a human lung adenocarcinoma cell line harboring a KRAS^{G13D} mutation and was purchased from the German Collection of Microorganisms and Cell Cultures GmbH (DSMZ, #ACC 234). The xA-427 cell line, utilized in the described in vivo study, was derived from in vivo passaged tumors collected from the mice harboring A-427 tumors. Tumor cells were isolated from the A-427 tumor tissues and in vitro passaged to generate the in vivo model. A-427 and xA-427 cells were cultured in medium containing EMEM plus 10% Fetal Bovine Serum (FBS) and 1% Antibiotic-Antimycotic (AA) at 37° C. in an atmosphere of 5% CO₂ in air. The medium was renewed every 2 to 3 days and tumor cells were routinely sub-cultured twice weekly at a confluence of 80-90% by trypsin-EDTA. The cells growing in an exponential growth phase were harvested and counted for inoculation. The xA-427 model is also referred to as A-427. A-427 tumor cells (passage 16) were implanted into mice subcutaneously. 200 μL cell suspensions containing 10×10⁶ tumor cells mixed with 50% Matrigel were subcutaneously implanted into the right flank of mouse using a syringe. When tumor volumes reached a mean of 210 mm³ (range of 140-288 mm3), tumor-bearing mice were randomized into different groups with 8 mice in each group. The randomization date was denoted as treatment day 0.

### Treatment

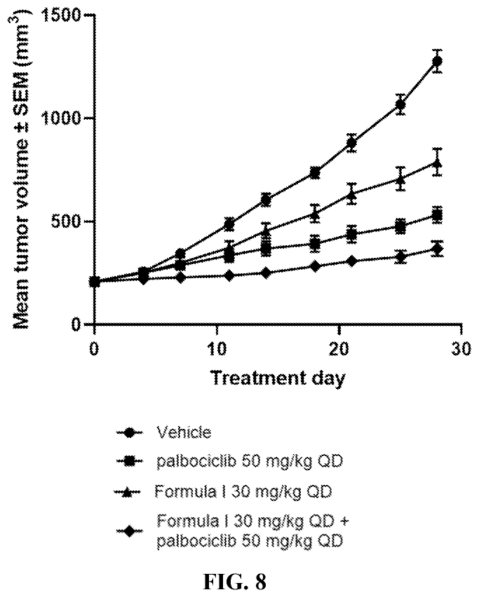

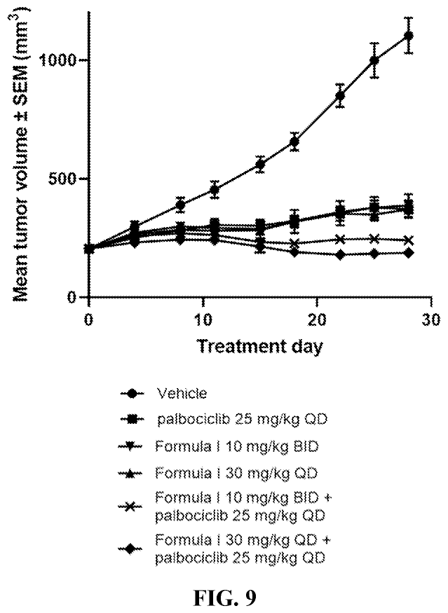

Treatment started on the day of randomization. The treatment start day was denoted as treatment day 0. Mice were dosed by oral administration of vehicle control solution, palbociclib at 50 mg/kg QD, Formula I at 30 mg/kg QD monotherapy treatment groups. One additional group received combination treatment of Formula I and palbociclib, Formula I at 30 mg/kg QD with palbociclib at 50 mg/kg QD. Palbociclib was dosed at one-hour post Formula I QD dose in combination groups. The study was terminated on treatment day 28 as defined in the study protocol. The results are shown in FIG. 8.

### Example 4. Combination Efficacy of the Compound of Formula I and Palbociclib in KRAS^{G12D} NSCLC PDX LUN137

### Vehicle/Control Article

The vehicle/control article, 100 mM acetic acid in deionized water, with pH adjustment to 4.8-5.0, was prepared and stored under ambient conditions throughout the 28-day administration in mice.

### Formulation of Test Article

The test article Formula I was prepared in vehicle of 100 mM acetic buffer weekly and stored under ambient conditions. The combination agent palbociclib was prepared in vehicle of 50 mM Sodium Lactate Buffer, pH 3.8-4.0 weekly and stored at 2-8° C.

### Animals

Female Balb/c nude mice were hosted at special pathogen-free (SPF) environment of vivarium facility and acclimated to their new environment for at least 3 days prior to initiation of any experiments. Mice were between 6-8 weeks of age at the time of implantation.

### Preparation of Xenograft Model

The LUN137 PDX model was established for pre-clinical efficacy study at GenenDesign (Shanghai, China). This PDX model was derived from a 57-year-old male Chinese NSCLC patient. The KRAS^{G12D} mutation in the PDX model LUN137 was confirmed by whole exome sequencing and PCR sequencing. Tumor fragments harvested from the PDX model were implanted subcutaneously in the right flanks of female Balb/c nude mice. Mice were anesthetized with isoflurane and anesthesia was maintained throughout the implantation procedure. Mouse skin was cleaned with appropriate surgical scrub and alcohol over the right flank. Aseptic surgical procedures were used. A small skin incision was made using the sharp end of the trochar and a 1.5 cm subcutaneous pocket along the right lateral chest wall was formed by blunt dissection with the stylet of a 10-12 g trochar needle. Tumor fragments (15-30 mm³) were placed into the trochar needle and advanced into the subcutaneous pocket in the right flank. Trochar incision was closed with suture or a wound clip that was removed one week after closure.

When tumor sizes reached 158-252 mm³ in volume, tumor-bearing mice were randomly divided into study groups with 8 mice in each group. The randomization date was denoted as treatment day 0.

### Treatment

Treatment started on the day of randomization. The treatment start day was denoted as treatment day 0. Mice were dosed by oral administration of vehicle control solution, Formula I at 10 mg/kg/dose BID, Formula I at 30 mg/kg QD, or palbociclib at 25 mg/kg QD monotherapy treatment groups. Two additional groups received combination treatments of Formula I and palbociclib, one dosing Formula I at 10 mg/kg/dose BID with palbociclib at 25 mg/kg QD and the other dosing Formula I at 30 mg/kg QD with palbociclib at 25 mg/kg QD. The dosing volume for each compound was 5 mL/kg and interval of BID regimen was 8 hours. Palbociclib was dosed at one-hour post Formula I QD or first BID dose in combination groups. The study was terminated on treatment day 28 as defined in the study protocol. The results are shown in FIG. 9.

### Example 5. Combination Efficacy of the Compound of Formula I and Palbociclib in KRAS^{G12D} CRC CDX GP2D

### Vehicle/Control Article

The vehicle/control article, 100 mM acetic acid in deionized water, with pH adjustment to 4.8-5.0, was prepared and stored under ambient conditions throughout the 28-day administration in mice.

### Formulation of Test Article

The test article Formula I was prepared in vehicle of 100 mM acetic buffer weekly and stored under ambient conditions. The combination agent palbociclib was prepared in vehicle of 50 mM Sodium Lactate Buffer, pH 3.8-4.0 weekly and stored at 2-8° C.

### Animals

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

### Preparation of Xenograft Model

GP2D cell line was human CRC harboring KRAS^{G12D} mutation and purchased from the European Collection of Authenticated Cell Cultures (ECACC, 95090714). GP2D cells were cultured in medium containing Dulbecco's Modified Eagle Medium (DMEM) plus 10% Fetal Bovine Serum (FBS) and 1% Antibiotic-Antimycotic (AA) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation. GP2D tumor cells were implanted into mice subcutaneously. 200 μL cell suspensions containing 10×10⁶ tumor cells mixed with 50% Matrigel were subcutaneously implanted into the right flank of mouse using a syringe. When tumor volumes reached a mean of 219 mm³ (range of 154-295 mm3), tumor-bearing mice were randomized into different groups with 8 mice in each group. The randomization date was denoted as treatment day 0.

### Treatment

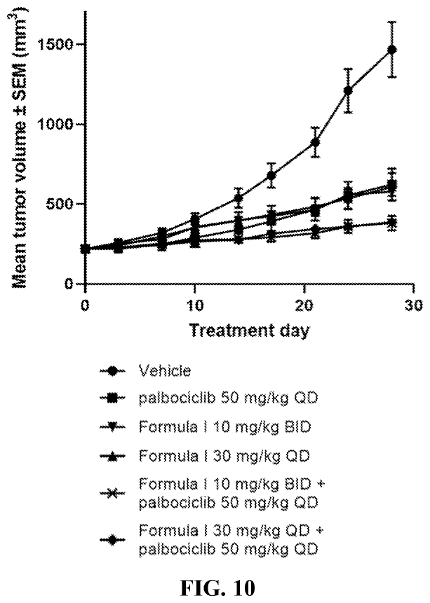

Treatment started on the day of randomization. The treatment start day was denoted as treatment day 0. Mice were dosed by oral administration of vehicle control solution, Formula I at 10 mg/kg/dose BID, Formula I at 30 mg/kg QD, or palbociclib at 25 mg/kg QD monotherapy treatment groups. Two additional groups received combination treatment of Formula I and palbociclib, one dosing Formula I at 10 mg/kg/dose BID with palbociclib at 25 mg/kg QD and the other dosing Formula I at 30 mg/kg QD with palbociclib at 25 mg/kg QD. The dosing volume for each compound was 5 mL/kg and interval of BID regimen was 8 hours. Palbociclib was dosed at one-hour post Formula I QD or first BID dose in combination groups. The study was terminated on treatment day 28 as defined in the study protocol. The results are shown in FIG. 10.

### Example 6. Combination Efficacy of the Compound of Formula I and Palbociclib in KRAS^{G12D} CRC CDX LS513

### Vehicle/Control Article

The vehicle/control article of Formula I, 100 mM acetic acid in deionized water, with pH adjustment to 4.8-5.0, was prepared and stored under ambient conditions throughout the 28-day administration in mice.

The vehicle/control article of palbociclib, 50 mM sodium lactate in deionized water, with pH adjustment to 3.8-4.0, was prepared and stored at 2-8° C. throughout the 28-day administration in mice.

### Formulation of Test Article

The test article Formula I was prepared in vehicle of 50 mM acetic buffer weekly and stored under ambient conditions. The combination agent palbociclib was prepared weekly in vehicle of 50 mM sodium lactate buffer, pH 3.8-4.0, and stored at 2-8° C.

### Animals

Female Balb/c nude mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

### Preparation of Xenograft Model

LS513 was a human colorectal carcinoma (CRC) cell line that harbored a KRAS^{G12D} mutation. The LS513 cell line was purchased from the American Type Culture Collection (ATCC® CRL-2134™) LS513 cells were cultured in medium containing RPMI1640 plus 10% Fetal Bovine Serum (FBS) and 1% Antibiotic-Antimycotic (AA) at 37° C. in an atmosphere of 5% CO₂ in air. The medium was renewed every 2 to 3 days and tumor cells were routinely sub-cultured twice weekly at a confluence of 80-90% by trypsin-EDTA. The cells growing in an exponential growth phase were harvested and counted for inoculation.

LS513 tumor cells were implanted into mice subcutaneously. Briefly, 200 μL cell suspensions containing 5×10⁶ tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 196 mm³ (range of 103-269 mm³), tumor-bearing mice were randomized into different groups with 8 mice in each group. The randomization date was denoted as treatment day 0.

### Treatment

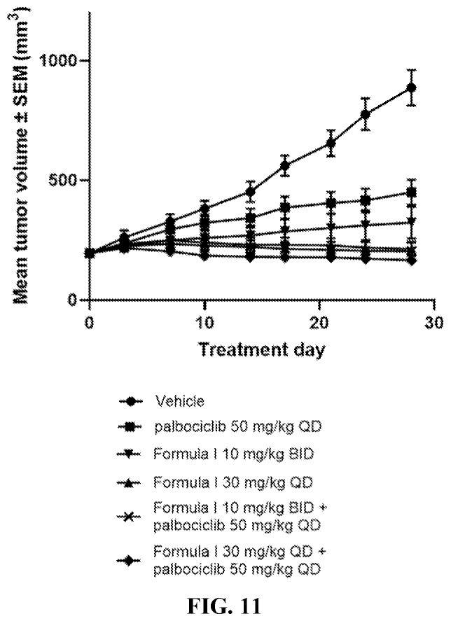

Treatment started on the day of randomization. The treatment start day was denoted as treatment day 0. Mice were dosed by oral administration of vehicle control solution, palbociclib at 50 mg/kg QD, Formula I at 10 mg/kg/dose BID, or Formula I at 30 mg/kg QD monotherapy treatment groups. Two additional groups received combination treatment of Formula I and palbociclib, one dosing Formula I at 10 mg/kg/dose BID with palbociclib at 50 mg/kg QD and the other dosing Formula I at 30 mg/kg QD with palbociclib at 50 mg/kg QD. The dosing volume for each compound was 5 mL/kg and interval of BID regimen was 8 hours. Palbociclib was dosed at one-hour post Formula I QD or first BID dose in combination groups. The study was terminated on treatment day 28 as defined in the study protocol. The results are shown in FIG. 11.

### Example 7. Combination Efficacy of the Compound of Formula I and Palbociclib in KRAS^{G12V} NSCLC CDX NCI-H441

### Vehicle/Control Article

The vehicle/control article, 100 mM acetic acid in deionized water, with pH adjustment to 4.8-5.0, was prepared and stored under ambient conditions throughout the 28-day administration in mice.

### Formulation of Test Article

The test article Formula I was prepared in vehicle of 100 mM acetic buffer weekly and stored under ambient conditions. The combination agent palbociclib was prepared in vehicle of 50 mM Sodium Lactate Buffer, pH 3.8-4.0 weekly and stored at 2-8° C.

### Animals

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of Xenograft Model

NCI-H441 was a human lung cancer cell line that harbored a KRAS^{G12V} mutation. The NCI-H441 cell line was purchased from the American Type Culture Collection (ATCC® HTB-174™). NCI-H441 cells were cultured in medium containing RPMI1640 plus 10% Fetal Bovine Serum (FBS) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation.

NCI-H441 tumor cells were implanted into mice subcutaneously. Briefly, 200 μL cell suspensions containing 5×10⁶ tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 196 mm³ (range of 150-251 mm³), tumor-bearing mice were randomized into different groups with 8 mice in each group. The randomization date was denoted as treatment day 0.

### Treatment

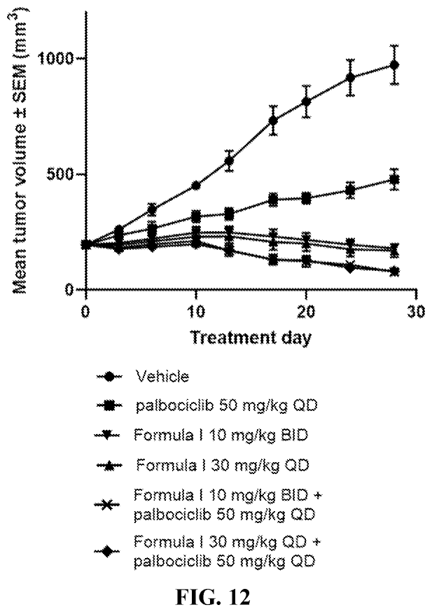

Treatment started on the day of randomization. The treatment start day was denoted as treatment day 0. Mice were dosed by oral administration of vehicle control solution, Formula I at 10 mg/kg/dose BID, Formula I at 30 mg/kg QD, or palbociclib at 50 mg/kg QD monotherapy treatment groups. Two additional groups received combination treatment of Formula I and palbociclib, one dosing Formula I at 10 mg/kg/dose BID with palbociclib at 50 mg/kg QD and the other dosing Formula I at 30 mg/kg QD with palbociclib at 50 mg/kg QD. The dosing volume for each compound was 5 mL/kg and interval of BID regimen was 8 hours. Palbociclib was dosed at one-hour post Formula I QD or first BID dose in combination groups. The study was terminated on treatment day 28 as defined in the study protocol. The results are shown in FIG. 12.

Although the foregoing embodiments have been described in some detail by way of illustration and Examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### REFERENCES

1. Puyol, M. et al. A Synthetic Lethal Interaction between K-Ras Oncogenes and Cdk4 Unveils a Therapeutic Strategy for Non-small Cell Lung Carcinoma. Cancer Cell 18, 63-73 (2010).
2. Pek, M. et al. Oncogenic KRAS-associated gene signature defines co-targeting of CDK4/6 and MEK as a viable therapeutic strategy in colorectal cancer. Oncogene 36, 4975-4986 (2017).
3. Sweeney, S. M. et al. AACR project genie: Powering precision medicine through an international consortium. Cancer Discovery 7, 818-831 (2017).
4. Hunter, J. C. et al. Biochemical and structural analysis of common cancer-associated KRAS mutations. Molecular Cancer Research 13, 1325-1335 (2015).
5. Nichols, R. J. et al. RAS nucleotide cycling underlies the SHP2 phosphatase dependence of mutant BRAF-, NF1- and RAS-driven cancers. Nature Cell Biology 20, 1064-1073 (2018).
6. Ostrem, J. M., Peters, U., Sos, M. L., Wells, J. A. & Shokat, K. M. K-Ras(G12C) inhibitors allosterically control GTP affinity and effector interactions. Nature 503, 548-551 (2013).
7. Allele-specific inhibitors inactivate mutant KRAS G12C by a trapping mechanism Science.
8. Hallin, J. et al. The KRASG12C inhibitor MRTX849 provides insight toward therapeutic susceptibility of KRAS-mutant cancers in mouse models and patients. Cancer Discovery 10, 54-71 (2020).
9. Mainardi, S. et al. SHP2 is required for growth of KRAS-mutant non-small-cell lung cancer in vivo letter. Nature Medicine 24, 961-967 (2018).
10. Chen, Y. N. P. et al. Allosteric inhibition of SHP2 phosphatase inhibits cancers driven by receptor tyrosine kinases. Nature 535, 148-152 (2016).
11. Hamilton and Infante. Targeting CDK4/6 in patients with cancer. Cancer Treatment Reviews 45, 129-138 (2016).

What is claimed is:

1. A method of treating a subject having cancer comprising administering to the subject a therapeutically effective amount of a compound of Formula I or its pharmaceutically acceptable salt: 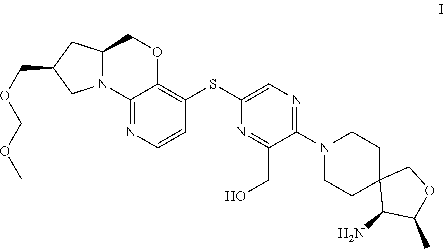 [SMILES: COCOC[C@H]1C[C@H]2COc3c(Sc4cnc(N5CCC6(CC5)CO[C@@H](C)[C@H]6N)c(CO)n4)ccnc3N2C1]
in combination with an inhibitor of cyclin D-cyclin dependent kinase (CDK) 4/6.

2. The method of claim 1, wherein the cancer is characterized by a mutation in KRAS.

3. A method of treating a subject having cancer comprising:
a) selecting a patient having a cancer characterized by a mutation in the cyclin D-cyclin dependent kinase (CDK) 4/6 pathway; and
b) administering to the subject a therapeutically effective amount of a compound of Formula I or its pharmaceutically acceptable salt: 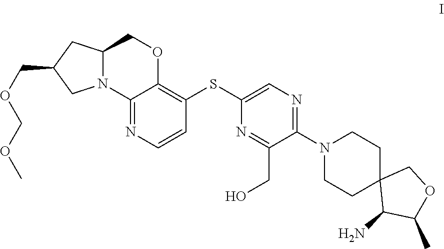 [SMILES: COCOC[C@H]1C[C@H]2COc3c(Sc4cnc(N5CCC6(CC5)CO[C@@H](C)[C@H]6N)c(CO)n4)ccnc3N2C1]
in combination with an inhibitor of CDK4/6.

4. The method of claim 3, wherein the mutation in the CDK4/6 pathway comprises a mutation in KRAS.

5. The method of claim 2 or 4, wherein the mutation in KRAS is G12D, G12S, G12C, G12V, G13D, Q61H, Q61K, or Q61R.

6. The method of any one of claims 1 to 5, wherein the cancer is colorectal cancer, non-small cell lung cancer, head and neck cancer, endometrial carcinoma, pancreatic cancer, melanoma, head and neck squamous cell carcinoma, liposarcoma, or neuroblastoma.

7. The method of claim 6, wherein the cancer is colorectal cancer.

8. The method of claim 6, wherein the cancer is non-small cell lung cancer.

9. The method of any one of claims 1 to 8, wherein the CDK4/6 inhibitor is selected from the group consisting of palbociclib, ribociclib, abemaciclib, FCN-437c, and alvociclib.

10. The method of any one of claims 1 to 8, wherein the CDK4/6 inhibitor is palbociclib.

11. The method of claim 10, wherein palbociclib is administered in an amount that is between about 50 mg/day to about 500 mg/day.

12. The method of claim 10, wherein palbociclib is administered in an amount that is about 75 mg/day, about 100 mg/day, about 125 mg/day, or about 150 mg/day.

13. The method of claim 10, wherein palbociclib is administered in an amount that is between about 50 mg once a week and about 650 mg once a week.

14. The method of claim 13, wherein palbociclib is administered in an amount that is about 200 mg once a week, 300 mg once a week, 400 mg once a week, 500 mg once a week, or 600 mg once a week.

15. The method of any one of claims 1 to 14, wherein the CDK4/6 inhibitor is ribociclib.

16. The method of any one of claims 1 to 14, wherein the CDK4/6 inhibitor is abemacicl.

17. The method of any one of claims 1 to 14, wherein the CDK4/6 inhibitor is FCN-437c.

18. The method of any one of claims 1 to 14, wherein the CDK4/6 inhibitor is alvociclib (flavopiridol).

19. The method of any one of claims 1 to 18, wherein the method comprises administering a third MAPK pathway inhibitor.

20. The method of claim 19, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK2 inhibitor, FLT3 inhibitor, or ERK1/2 inhibitor.

21. The method of any one of claims 1 to 20, wherein the compound of Formula I is administered once or twice daily.

22. The method of any one of claims 1 to 21, wherein CDK4/6 inhibitor is administered once or twice daily.

23. The method of any one of claims 1 to 22, wherein the compound of Formula I is administered orally.

24. The method of any one of claims 1 to 23, wherein the dosing of the compound of Formula I is in a range from 20 mg to 400 mg daily.

25. The method of any one of claims 1 to 23, wherein the compound of Formula I is administered QD or BID for 2 weeks on and 1 week off (21 day schedule).

26. The method of any one of claims 1 to 23, wherein the compound of Formula I is administered QD or BID for 3 weeks on and 1 week off (28 day schedule).

27. The method of any one of claims 1 to 23, wherein the compound of Formula I is administered QD or BID three times a week (DID3D5 TIW) e.g., Day 1, Day 3, and Day 5.

28. The method of any one of claims 1 to 23, wherein the compound of Formula I is administered twice a day/twice a week e.g., Day 1 and Day 2 (BID-D1D2-BIW).

29. The method of any one of claims 1 to 23, wherein the compound of Formula I is administered once a day (QD) continuous dosing at a dose of 20 mg/day to 60 mg/day, 40 mg/day, or 60 mg/day.

30. The method of any one of claims 1 to 23, wherein the compound of Formula I is administered twice a day (BID) continuous dosing at a dose of 20 mg/day to 80 mg/day.

31. The method of any one of claims 1 to 23, wherein the compound of Formula I is administered twice a day (BID) continuous dosing at a dose of 10 mg/day to 100 mg/day.

32. The method of any one of claims 1 to 31, wherein the dosing of the CDK4/6 inhibitor is in a range from 1 mg to 1000 mg daily.

33. The method of any one of claims 1 to 32, further comprising administering a selective estrogen receptor degrader (SERD) or an aromatase inhibitor.

34. A method of treating colorectal cancer in a subject comprising orally administering to the subject a therapeutically effective amount of a compound of Formula I or its pharmaceutically acceptable salt: 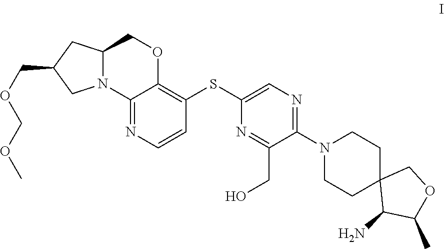 [SMILES: COCOC[C@H]1C[C@H]2COc3c(Sc4cnc(N5CCC6(CC5)CO[C@@H](C)[C@H]6N)c(CO)n4)ccnc3N2C1]
in combination with a CDK4/6 inhibitor.

35. The method of claim 34, wherein the cancer is characterized by a mutation in KRAS.

36. f claim 35, wherein the mutation in KRAS is G12D, G12S, G12C, G12V, G13D, Q61H, Q61K, or Q61R.

37. The method of any one of claims 34 to 36, wherein the CDK4/6 inhibitor is selected from the group consisting of palbociclib, ribociclib, abemaciclib, FCN-437c, and alvociclib.

38. The method of any one of claims 34 to 37, wherein the method comprises administering a third MAPK pathway inhibitor.

39. The method of any one of claims 34 to 38, wherein the compound of Formula I is administered once or twice daily.

40. The method of any one of claims 34 to 39, wherein CDK4/6 inhibitor is administered once or twice daily.

41. The method of any one of claims 34 to 40, wherein the compound of Formula I is administered orally.

42. The method of any one of claims 34 to 41, wherein the dosing of the compound of Formula I is in a range from 20 mg to 400 mg daily.

43. The method of any one of claims 34 to 41, wherein the compound of Formula I is administered QD or BID for 2 weeks on and 1 week off (21 day schedule).

44. The method of any one of claims 34 to 41, wherein the compound of Formula I is administered QD or BID for 3 weeks on and 1 week off (28 day schedule).

45. The method of any one of claims 34 to 41, wherein the compound of Formula I is administered QD or BID three times a week (D1D3D5 TIW) e.g., Day 1, Day 3, and Day 5.

46. The method of any one of claims 34 to 41, wherein the compound of Formula I is administered twice a day/twice a week e.g., Day 1 and Day 2 (BID-D1D2-BIW).

47. The method of any one of claims 34 to 41, wherein the compound of Formula I is administered once a day (QD) continuous dosing at a dose of 20 mg/day to 60 mg/day, 40 mg/day, or 60 mg/day.

48. The method of any one of claims 34 to 41, wherein the compound of Formula I is administered twice a day (BID) continuous dosing at a dose of 20 mg/day to 80 mg/day.

49. The method of any one of claims 34 to 41, wherein the compound of Formula I is administered twice a day (BID) continuous dosing at a dose of 10 mg/day to 100 mg/day.

50. The method of any one of claims 34 to 49, wherein the dosing of the CDK4/6 inhibitor is in a range from 1 mg to 1000 mg daily.

51. The method of any one of claims 34 to 50, further comprising administering a selective estrogen receptor degrader (SERD) or an aromatase inhibitor.

52. A method of treating a subject having cancer comprising administering to the subject a therapeutically effective amount of a compound of Formula I or its pharmaceutically acceptable salt: 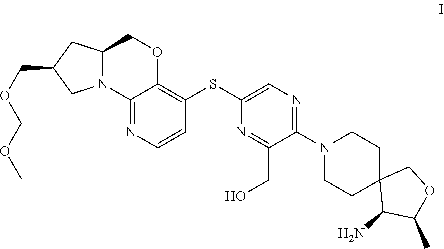 [SMILES: COCOC[C@H]1C[C@H]2COc3c(Sc4cnc(N5CCC6(CC5)CO[C@@H](C)[C@H]6N)c(CO)n4)ccnc3N2C1]
in combination with palbociclib.

53. A method of treating a subject having cancer comprising administering to the subject a therapeutically effective amount of a compound of Formula I or its pharmaceutically acceptable salt: 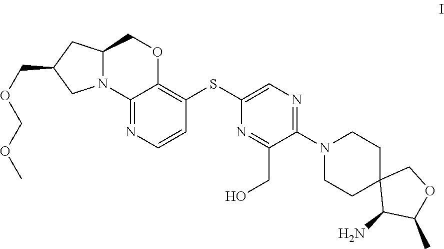 [SMILES: COCOC[C@H]1C[C@H]2COc3c(Sc4cnc(N5CCC6(CC5)CO[C@@H](C)[C@H]6N)c(CO)n4)ccnc3N2C1]
in combination with ribociclib.

54. A method of treating a subject having cancer comprising administering to the subject a therapeutically effective amount of a compound of Formula I or its pharmaceutically acceptable salt: 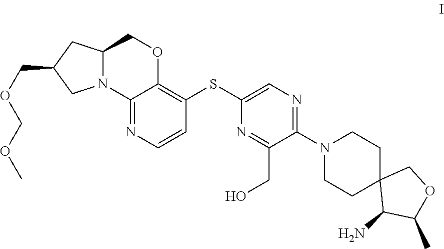 [SMILES: COCOC[C@H]1C[C@H]2COc3c(Sc4cnc(N5CCC6(CC5)CO[C@@H](C)[C@H]6N)c(CO)n4)ccnc3N2C1]
in combination with abemaciclib.

55. A method of treating a subject having cancer comprising administering to the subject a therapeutically effective amount of a compound of Formula I or its pharmaceutically acceptable salt: 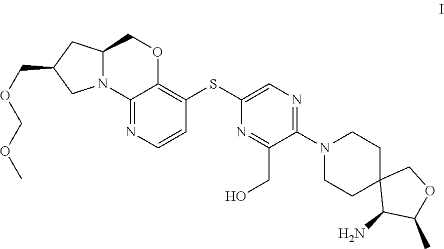 [SMILES: COCOC[C@H]1C[C@H]2COc3c(Sc4cnc(N5CCC6(CC5)CO[C@@H](C)[C@H]6N)c(CO)n4)ccnc3N2C1]
in combination with FCN-437c.

56. A method of treating a subject having cancer comprising administering to the subject a therapeutically effective amount of a compound of Formula I or its pharmaceutically acceptable salt: 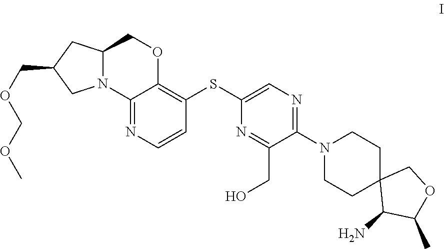 [SMILES: COCOC[C@H]1C[C@H]2COc3c(Sc4cnc(N5CCC6(CC5)CO[C@@H](C)[C@H]6N)c(CO)n4)ccnc3N2C1]
in combination with alvociclib.

57. The method of any one of claims 52-56, wherein the cancer is colorectal cancer, non-small cell lung cancer, head and neck cancer, endometrial carcinoma, pancreatic cancer, melanoma, head and neck squamous cell carcinoma, liposarcoma, or neuroblastoma.

58. The method of claim 57, wherein the cancer is colorectal cancer.

59. The method of claim 57, wherein the cancer is non-small cell lung cancer.

60. The method of claim 57, wherein the cancer is melanoma.

61. The method of claim 57, wherein the cancer is breast cancer.

62. The method of claim 57, wherein the cancer is a pan-tumor.

63. The method of any one of claims 1 to 62, wherein the subject is a human.

64. The method of any one of claims 1 to 63, wherein a dosing of the CDK4/6 inhibitor is less than a dosing required for a monotherapy with the CDK4/6 inhibitor, or less than a dosing required for a co-therapy with the CDK4/6 inhibitor and an aromatase inhibitor or a SERD.

65. The method of any one of claims 1 to 64, wherein a dosing of the compound of Formula I is less than a dosing required for a monotherapy with the compound of Formula I.

66. A kit comprising a compound of Formula I or its pharmaceutically acceptable salt: 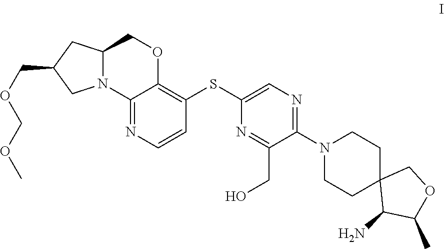 [SMILES: COCOC[C@H]1C[C@H]2COc3c(Sc4cnc(N5CCC6(CC5)CO[C@@H](C)[C@H]6N)c(CO)n4)ccnc3N2C1]
and a CDK4/6 inhibitor.

67. The kit of claim 66, wherein the compound of Formula 1 and the CDK4/6 inhibitor are in separate packages.

68. The kit of claim 66 or 67, wherein the CDK4/6 inhibitor is one or more of palbociclib, ribociclib, abemaciclib, FCN-437c, and alvociclib.

69. The kit of any one of claims 66 to 68, further comprising an aromatase inhibitor or a SERD.

70. The kit of any one of claims 66 to 69, wherein the kit further comprises instructions to administer the contents of the kit to a subject for the treatment of cancer.
